(19) United States
(12) Patent Application Publication
(10) Pub. No.: US 2011/0003883 A1
(43) Pub. Date: Jan. 6, 2011

(54) ALLOSTERIC TRANS-SPLICING GROUP I RIBOZYME WHOSE ACTIVITY OF TARGET-SPECIFIC RNA REPLACEMENT IS CONTROLLED BY THEOPHYLLINE

(75) Inventors: Seong Wook Lee, Seoul (KR); Sun Young Jang, Ulsan (KR); Ju Hyun Kim, Gyeonggi-do (KR)

Correspondence Address: MERCHANT & GOULD PC, P.O. BOX 2903, MINNEAPOLIS, MN, 55402-0903, US

(73) Assignee: Industry-Academic Cooperation Foundation, Dankook University, Gyeonggi-do (KR)

(21) Appl. No.: 12/442,258

(22) PCT Filed: Dec. 16, 2008
(86) PCT No.: PCT/KR08/07440

§ 371 Date: Mar. 20, 2009

(30) Foreign Application Priority Data
Mar. 27, 2008 (KR) 10-2008-0028483

(51) Int. Cl. A61K 48/00; C12Q 1/68; C12N 15/63; C07H 21/02

(52) U.S. Cl. 514/44 R; 435/6; 435/320.1; 536/23.1; 536/23.5; 536/23.2

(57) ABSTRACT

Provided is an allosteric trans-splicing group I ribozyme whose target-specific RNA replacement activity is controlled by theophylline, wherein the hTERT-targeting trans-splicing ribozyme recognizes mRNA of human telomerase reverse transcriptase (hTERT) as a cancer-specific RNA transcript to bind a theophylline aptamer to an hTERT target trans-splicing ribozyme via a communication module, the hTERT target trans-splicing ribozyme having a verified trans-splicing ability. The allosteric trans-splicing group I ribozyme may be useful to selectively diagnose only cancer cells that express target hTERT RNA, or induce their apoptosis since the activity of the allosteric trans-splicing group I ribozyme is dependently controlled by theophylline to correct target hTERT RNA by the trans-splicing reaction.

### CROSS REFERENCE TO RELATED CASES

This application is a 371 National Stage of PCT/KR2008/007440, filed Dec. 16, 2008, which claims priority from Korean Patent Application No. 10-2008-0028483, filed Mar. 27, 2008, the entire disclosures of which are incorporated herein by reference in their entirety.

### TECHNICAL FIELD

The present invention relates to an allosteric trans-splicing group I ribozyme whose target-specific RNA replacement activity is controlled by theophylline.

### BACKGROUND ART OF THE INVENTION

There have been ardent attempts to develop a gene therapy technology as a new therapy technology to treat incurable human diseases by studying the molecular genetic causes and factors of the incurable human diseases caused by the gene mutations. However, there are many problems to be solved in the existing gene therapy technologies.

Gene therapy that has been widely used to treat genetic diseases is devised by transferring a normal gene corresponding to a mutant gene to patient's proper cells (Morgan, R. A. and Anderson, W. F. 1993, Human gene therapy. Ann. Rev. Biochem. 62: 191-217). Theoretically in order to obtain therapeutic effects through this gene therapy, desired gene products should be produced under a proper in vivo control mechanism. Due to the limits on the sizes of transporter genes in virus particles, however, nearly all of the gene therapies are devised by transferring a desired gene in the form of cDNA under the control of a variety of different promoters or of the genes' own promoters in their fragments. Therefore, the virus particles do not include a variety of genetic elements that may control transporter genes in and of themselves, and do not maximize the desired effects for the treatment diseases.

Also, the promoters used for gene expression and the like may undesirably activate other kinds of promoters, and also may increase the expression of different genes (for example, protooncogenes) of cells, to which the genes are transferred, by changing the chromatin structure. Also, the transfer of normal genes does not affect the decrease in mutant gene products in patient's cells at all. When the mutant gene products do have a dominant negative effect, their therapeutic effects may not be maximized by the conventional methods. Therefore, there is a demand for a novel gene therapy that induces the well-controlled expression of the normal genes and suppresses the expression of mutant genes at the same time (Lan, N., Howrey, R. P., Lee, S. W., Smith, C. A., and Sullenger, B. A. 1998, Ribozyme-Mediated Repair of Sickle b-Globin mRNAs in Erythrocyte Precursors. Science 280: 1593; Phylactou, L. A., Darrah, C., and Wood, M. J. 1998, Ribozyme-mediated trans-splicing of a trinucleotide repeat. Nat. Genet. 18: 378-381; Rogers, C. S., Vanoye, C. G., Sullenger, B. A., and George, A. L. Jr. 2002, Functional repair of a mutant chloride channel using a trans-splicing ribozyme, J. Clin. Invest. 110: 1783-1789; Shin, K. S., Sullenger, B. A., and Lee, S. W. 2004, Ribozyme-mediated induction of apoptosis in human cancer cells by targeted repair of mutant p53 RNA. Mol. Ther. 10: 365-372; Ryu, K. J., Kim, J. H., and Lee, S. W. 2003, Ribozyme-mediated selective induction of new gene activity in hepatitis C virus internal ribosome entry site-expressing cells by targeted trans-splicing. Mol. Ther. 7; 386-395).

It was reported that group I intron ribozyme from Tetrahymena thermophila connect two separate transcripts to each other by trans-splicing the separate transcripts in bacterial cells and human cells as well as in vitro conditions (Been, M. and Cech, T. 1986, One binding site determines sequence specificity of Tetrahymena pre-rRNA self-splicing, trans-splicing, and RNA enzyme activity. Cell 47: 207-216; Sullenger, B. A. and Cech, T. R. 1994, Ribozyme-mediated repair of defective mRNA by targeted, trans-splicing. Nature 371: 619-622; Jones, J. T., Lee, S. W., and Sullenger, B. A. 1996, Tagging ribozyme reaction sites to follow trans-splicing in mammalian cells. Nat. Med. 2: 643-648). Therefore, the trans-splicing ribozyme that functions on the basis of the group I intron targets disease-associated gene transcripts, or certain RNAs that are not expressed in normal cells but are specifically expressed in infected cells, and then induces re-programming of the cells by correcting the abnormal RNA into normal RNA or substituting the disease-associated gene transcripts with new therapeutic gene transcripts. Accordingly, the trans-splicing ribozyme is very specific to diseases and may be used for a stable gene therapy technology. That is to say, since the RNA replacement is performed under the mere presence of target gene transcripts, desired gene products may be produced only in a proper space at a proper time. In particular, since the RNA replacement is used to target intracellularly expressed RNA and then substitute the targeted RNA with a desired gene product, it is possible to control an amount of the expressed genes to be introduced. Also, the trans-splicing ribozyme may double the therapeutic effects since it functions to remove the disease-specific RNA and simultaneously induce the expression of desired therapeutic gene products.

RNA has suitable chemical and structural characteristics to function as an artificial or natural switch (Mandal, M., Boese, B., Barrick, J. E., Winkler, W. C., and Breaker, R. R. 2003, Riboswitches control fundamental biochemical pathways in Bacillus subtilis and other bacteria. Cell 113: 577-586). By employing these characteristics, an enzyme, which is obtained by recognizing a certain structure or sequence of a small molecule or protein to specifically bind an RNA aptamer to a ribozyme that is an RNA having an enzyme activity, is referred to as an aptazyme (Breaker, R.R. 2002, Engineered allosteric ribozymes as biosensor components. Curr. Opin. Biotechnol. 13: 31-39). For the aptazyme, the ribozyme and the aptamer are connected by means of a communication module. The communication module has a structure that functions as an intermediate that transfers signals generated in the aptamer to the ribozyme (Kertsburg, A. and Soukup, G. A. 2002, A versatile communication module for controlling RNA folding and catalysis. Nucleic Acids Res. 30: 4599-4606).

When a ligand is sensed by the aptamer, these signals are transferred to the ribozyme via the communication module to allosterically modify an inert ribozyme in order to induce or suppress the activities of the ribozyme. That is to say, the activity of the ribozyme may be controlled by a certain endogenous or exogenous ligand.

For the recent methods of treating cancer, there is a demand for developing a method where only cancer cells can be specifically removed. Allosteric ribozyme (aptazyme) is prepared by binding an RNA aptamer to a ribozyme by using the fact that a structure of the ribozyme is changed by the binding of RNA to other ligands, etc. An exact mechanism of the allosteric ribozyme using small molecules as the ligand has not been known, but it is considered that the mechanism of the allosteric ribozyme is performed by binding to a ligand to structurally stabilize or destabilize the ribozyme (Kertsburg, A. and Soukup, G. A. 2002, A versatile communication module for controlling RNA folding and catalysis. Nucleic Acids Res. 30: 4599-4606; Jose, A. M., Soukup, G. A., and Breaker, R.R. 2001, Cooperative binding of effectors by an allosteric ribozyme. Nucleic Acids Res. 29: 1631. 1637; Koizumi, M., Soukup, G. A., Kerr, J. N., and Breaker, R. R. 1999, Allosteric selection of ribozymes that respond to the second messengers cGMP and cAMP. Nature Struct. Biol. 6: 1062.1071). The aptazyme using the small molecule as the ligand has been studied at an early stage, but the studies on aptazyme have been carried recently which interacts with proteins or oligos.

Human telomerase reverse transcriptase (hTERT) is one of factors to control the immortality and proliferation of cancer cells. The telomerase has 80 to 90% telomerase activity in endlessly reproduced germ cells, hematopoietic cells and cancer cells, but normal cells surrounding the cancer cells do not have this activity (Bryan, T. M. and Cech, T. R. 1999, Telomerase and the maintenance of chromosome ends. Curr. Opin. Cell Biol. 11; 318-324). By using theses characteristics of the telomerase, there have been ardent attempts to develop an inhibitor of the telomerase associated with cell growth in order to suppress the proliferation of the cancer cells (Bryan, T. M., Englezou, A., Gupta, J., Bacchetti, S., and Reddel, R. R. 1995, Telomere elongation in immortal human cells without detectable telomerase activity. EMBO J. 14; 4240-4248; Artandi, S. E. and DePinho, R. A. 2000, Mice without telomerase: what can they teach us about human cancer Nat. Med. 6; 852-855).

The present inventors have found a variety of theophylline-dependent allosteric trans-splicing ribozymes that are prepared by specifically recognizing RNA of cancer cell-specific human telomerase reverse transcriptase (hTERT) and bind a hTERT-targeting trans-splicing ribozyme to an aptamer by means of a commercialized communication module, wherein the hTERT-targeting trans-splicing ribozyme has a verified trans-splicing ability, and the aptamer has a high affinity to theophylline.

Also, the present invention has confirmed that theses ribozymes selectively recognize and cleave hTERT RNA only in a condition where theophylline is present in a test tube and cells, and anneal 3′ exon of the ribozyme to a downstream region of a target site, by using an in vitro trans-splicing assay, a luciferase assay, RT-PCR and an MTT assay.

These allosteric trans-splicing ribozymes may be used to develop a system that is able to target certain disease-specific RNA and artificially control the replacement into therapeutic gene RNA by using exogenous factors such as small molecules to activate the functions of the ribozyme. Also, a novel concept of specific and reversible gene therapy technologies may be developed by artificially controlling the expression of therapeutic genes in an infected cell-specific manner (FIG. 1).

### SUMMARY OF THE INVENTION

The present invention is designed to solve the problems of the prior art, and therefore it is an object of the present invention to provide a method for selecting an allosteric trans-splicing group I ribozyme whose activity is controlled by theophylline.

Also, it is another object of the present invention to provide an allosteric trans-splicing group I ribozyme whose RNA replacement activity is controlled by theophylline and which specifically targets RNA of human telomerase reverse transcriptase (hTERT), and its use.

Furthermore, it is still another object of the present invention to provide an expression vector expressing the allosteric trans-splicing group I ribozyme, and its use.

According to an aspect of the present invention, there is provided a method for selecting an allosteric trans-splicing group I ribozyme whose activity is controlled by theophylline.

According to another aspect of the present invention, there are also provided an allosteric trans-splicing group I ribozyme whose RNA replacement activity is controlled by theophylline and which specifically targets RNA of human telomerase reverse transcriptase (hTERT), and its use.

According to still another aspect of the present invention, there are also provided an expression vector expressing the allosteric trans-splicing group I ribozyme, and its use.

As described above, the allosteric trans-splicing group I ribozyme according to one exemplary embodiment of the present invention may be useful to selectively diagnose only cancer cells that express target hTERT RNA, or induce their apoptosis since the activity of the allosteric trans-splicing group I ribozyme is dependently controlled by theophylline to correct target hTERT RNA by the trans-splicing reaction.

### BRIEF DESCRIPTION OF THE DRAWINGS

FIG. 1 is a schematic view showing the control of replacement into RNAs by an allosteric trans-splicing ribozyme.

FIG. 2 shows an hTERT targeting T/S ribozyme.

FIG. 3 shows a theophylline-dependent allosteric T/S ribozyme.

FIG. 4 shows 3′ end sequences of WT P9 and Mu-P9.

FIG. 5 shows an in vitro trans-splicing reaction.

FIG. 6 shows a real-time PCR assay of in vitro trans-splicing reaction products.

FIG. 7 shows an in vitro trans-splicing reaction by a T/S ribozyme having an extended intergenic spacer (IGS).

FIG. 8 shows compatibility of the in vitro trans-splicing reaction by an allosteric trans-splicing ribozyme.

FIG. 9 shows induction of a theophylline-dependent transgene by an allosteric trans-splicing ribozyme.

FIG. 10 shows induction of a theophylline-dependent transgene by an allosteric trans-splicing ribozyme including a 100-nt anti-sense sequence against target RNA.

FIG. 11 shows suppression of a transgene by an allosteric trans-splicing ribozyme in hTERT cells.

FIG. 12 shows induction of a theophylline-dependent transgene by an allosteric trans-splicing ribozyme including a 300-nt anti-sense sequence against target RNA.

FIG. 13 shows a theophylline-dependent trans-splicing reaction by an allosteric ribozyme in cells.

FIG. 14 shows basic structures of an expression vector (pAvQ-Theo-Rib21AS-TK) and an adenovirus vector (Ad-TheoRib-TK, Ad-Theo-CRT) each encoding a theophylline-dependent trans-splicing ribozyme.

FIG. 15 shows theophylline-dependent cell apoptosis by an allosteric trans-splicing ribozyme in hTERT+HT-29 cells.

FIG. 16 shows theophylline-dependent cell apoptosis by an allosteric trans-splicing ribozyme in hTERT+HepG2 cells.

FIG. 17 shows theophylline-dependent cell apoptosis by an allosteric trans-splicing ribozyme in hTERT+Capan-1 cells.

FIG. 18 shows no cell apoptosis by an allosteric trans-splicing ribozyme in hTERT−IMR90 cells.

FIG. 19 shows a trans-splicing reaction of HT-29 cells by an allosteric trans-splicing ribozyme.

FIG. 20 shows a trans-splicing reaction of HT-29 cells by an allosteric trans-splicing ribozyme using a real-time PCR assay.

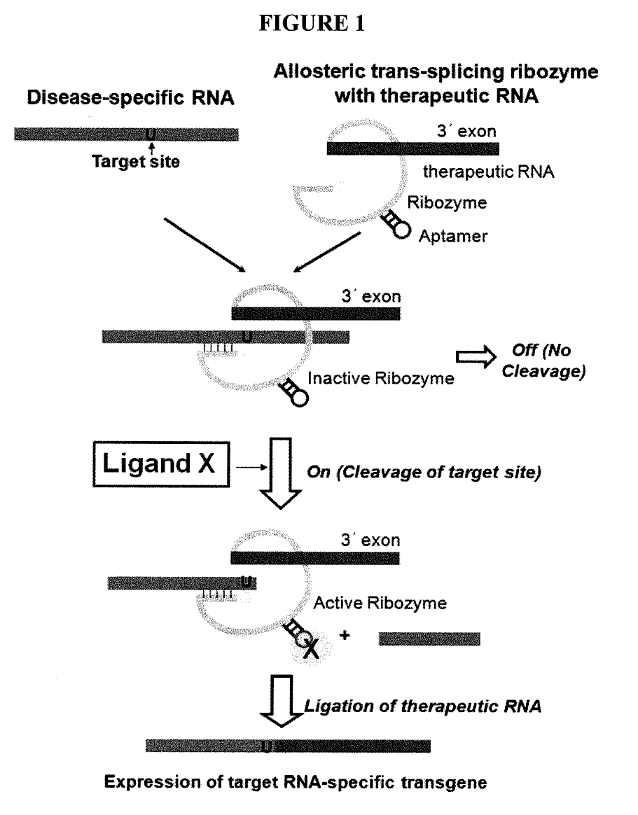

### DETAILED DESCRIPTION OF THE INVENTION

The present invention provides a method for selecting an allosteric trans-splicing group I ribozyme whose activity is controlled by theophylline, the method including: preparing an aptazyme where a theophylline aptamer and a communication module bind to either or both of P6 and P8 domains of a trans-splicing ribozyme, an aptazyme where a theophylline aptamer and a communication module bind to either or both of P6 and P8 domains of a trans-splicing ribozyme whose P9 domain is partially removed, or an aptazyme where a theophylline aptamer and a communication module bind to either or both of P6 and P8 domains of a trans-splicing ribozyme whose P9 domain is partially modified; confirming whether a theophylline-dependent trans-splicing reaction occurs by using theophylline and caffeine to compare the allosteric controls of the in vitro prepared aptazyme; and confirming whether a theophylline-dependent transgene is expressed at the presence of 0.1 to 1 mM theophylline by luciferase activity in mammalian cells.

In this case, the method for selecting an allosteric trans-splicing group I ribozyme according to one exemplary embodiment of the present invention may further include: preparing an aptazyme including an anti-sense 100 to 300 nt segment against hTERT RNA in the step of preparing an aptazyme.

Also, the present invention provides an allosteric trans-splicing group I ribozyme whose RNA replacement activity is controlled by theophylline, characterized in that the allosteric trans-splicing group I ribozyme specifically targets RNA of human telomerase reverse transcriptase (hTERT), and has a firefly-derived luciferase receptor gene at 3′ exon.

In this case, the allosteric trans-splicing group I ribozyme may have a RNA sequence selected from the group consisting of AS300 ΔP9 8T set forth in SEQ ID NO: 1, AS100 Mu-P9 6T8T set forth in SEQ ID NO: 2 and AS300 W-P9 6T8T set forth in SEQ ID NO: 3.

Also, the present invention provides an expression vector encoding the allosteric trans-splicing group I ribozyme.

In this case, the expression vector may include a vector selected from the group consisting of pSEAP AS300 Delta P9 8T-Luci set forth in SEQ ID NO: 4, pSEAP AS100 Mu-P9 6T8T-Luci set forth in SEQ ID NO: 5 and pSEAP AS300 W-P9 6T8T-Luci set forth in SEQ ID NO: 6.

Also, the present invention provides an allosteric trans-splicing group I ribozyme whose RNA replacement activity is controlled by theophylline, characterized in that the allosteric trans-splicing group I ribozyme specifically targets RNA of human Telomerase reverse transcriptase (hTERT), and has a herpes simplex virus thymidine kinase (HSV-TK) apoptosis gene at 3′ exon.

In this case, the allosteric trans-splicing group I ribozyme may have an RNA sequence of AS300 W-P9 6T8T-TK set forth in SEQ ID NO: 7.

Also, the present invention provides an expression vector expressing the allosteric trans-splicing group I ribozyme in mammalian cells.

In this case, the expression vector may include pAvQ-Theo-Rib21AS-TK (KCCM 10935P) set forth in SEQ ID NO: 8.

In addition, the present invention provides a gene expression inducer, cancer diagnostic agent or gene therapeutic agent including the allosteric trans-splicing group I ribozyme and theophylline.

Furthermore, the present invention provides a gene expression inducer, cancer diagnostic agent or gene therapeutic agent including the expression vector and theophylline.

Hereinafter, exemplary embodiments of the present invention will be described in more detail.

The allosteric trans-splicing group I ribozyme according to one exemplary embodiment of the present invention is referred to as an aptazyme or theophylline dependent aptazyme hereinafter.

The expression ‘theophylline aptamer’ used throughout this specification means an aptamer that specifically binds to theophylline.

The allosteric trans-splicing group I ribozyme according to one exemplary embodiment of the present invention is a molecule that may allosterically enhance or suppress the trans-splicing activity of a ribozyme due to structural changes in the ribozyme. Here, since a domain binding to a certain ligand such as an aptamer is annealed to a substrate binding site and a catalytic core site of the ribozyme, the structural changes in the ribozyme may be induced when an aptamer binds to the certain ligand and the ligand is sensed to transfer these signals to the ribozyme via a communication module.

The present inventors have found an aptazyme by binding a theophylline aptamer to an hTERT targeting trans-splicing ribozyme via a communication module. Here, the hTERT targeting trans-splicing ribozyme was previously developed on the basis of group I intron, and the activity of a trans-splicing ribozyme is controlled by theophylline and the aptazyme is also able to induce trans-splicing only in cancer cells having hTERT.

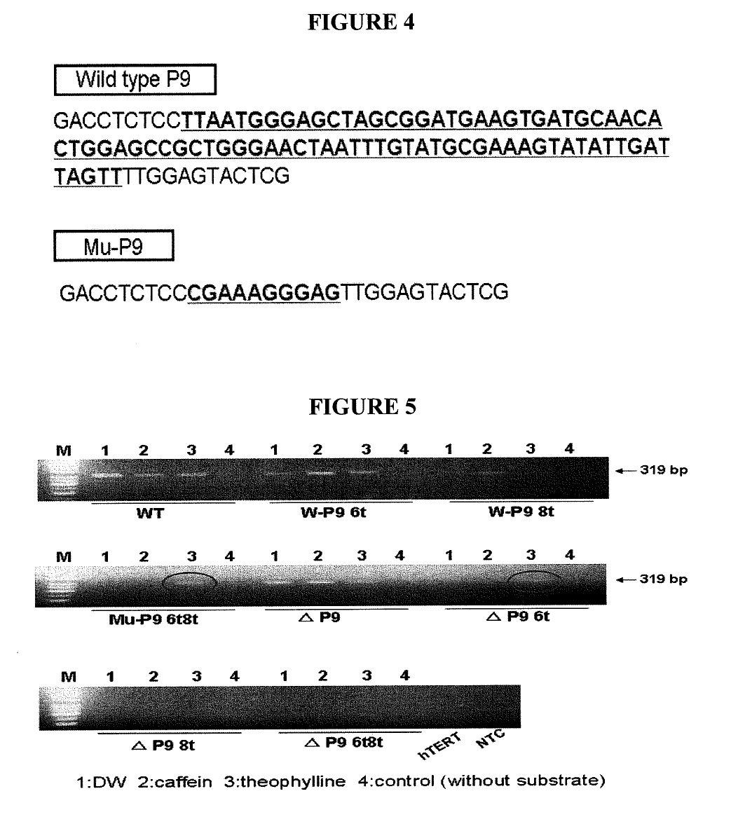

In this case, prepared was the aptazyme where a theophylline aptamer and a communication module bind to either or both of P6 and P8 domains of the trans-splicing ribozyme, or a theophylline aptamer and a communication module bind to either or both of P6 and P8 domains of a trans-splicing ribozyme whose P9 domain was partially removed (see FIG. 3). Here, the most commercialized communication module was used as a site at which the aptamer and the ribozyme are annealed with each other. Also, Mu-P9 6t8t where a partial DNA sequence of a P9 domain is substituted with a different sequence, was obtained in a PCR cloning procedure, and the construct was also used in experiments (see FIG. 4). All the experiments were carried out by comparing the results for theophylline, caffeine and an equivalent volume of a solvent (dH₂O or PBS). Here the caffeine having one different residue from the theophylline was used to confirm the experimental results for theophylline, and the solvent was used as control.

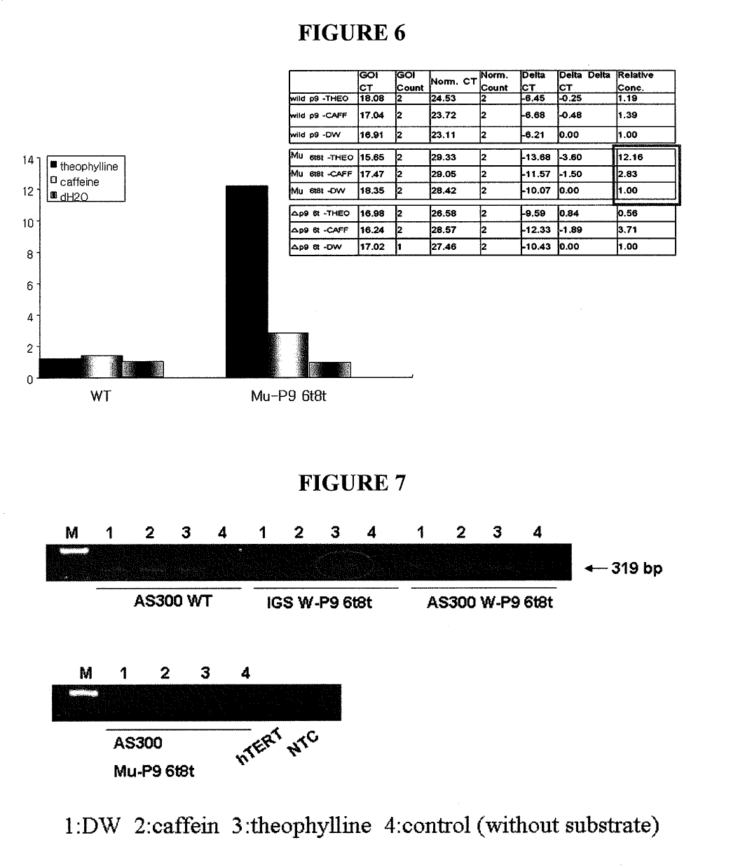

From the in vitro results of comparison and confirmation of the allosteric control of the aptazyme, it might be confirmed that Mu-P9 6t8t and ΔP9 6t are dependently trans-spliced by theophylline (see FIG. 5). Also, when the Mu-P9 6t8t and the ΔP9 6t were subject to a PCR reaction, a trans-splicing product of the Mu-P9 6t8t was expressed in a high amount of 40% or more, compared to that of the ΔP9 6t. By using a real-time PCR, it was confirmed that a trans-splicing product is generated in the Mu P9 6t8t 12 times higher when in the presence of theophylline than at the presence of dH2O (see FIG. 6). From the in vitro results of comparison and confirmation of the allosteric control of the aptazyme having extended IGS, it was also confirmed that, although the ribozyme has the same basic backbone, its activities are expressed differently according to the presence of an anti-sense sequence (see FIG. 7).

Also, the allosteric control of the aptazyme was determined in mammalian cells. Considering that the in vitro and intracellular allosteric control of the aptazyme is different from each other, the in vitro experimental results of the aptazyme were tested in vivo.

Before these experiments, cells were treated in a concentration-dependent manner so as to determine an optimum concentration of theophylline in the cells. As a result, it was determined that the optimum concentration of theophylline is preferably in a range of 0.1 to 1.0 mM, and more preferably 0.7 mM (see FIG. 9).

Then, a luciferase assay was performed so as to determine whether a trans-splicing product is expressed in cells and the expressed transgene is functional.

In the intracellular experiments, luciferase was expressed overall even at the presence of an equivalent volume of PBS (solvent) rather than theophylline or caffeine. This indicates that a luciferase gene present at a 3′ exon of the trans-splicing aptazyme is leakily expressed without trans-splicing. Therefore, when the ribozyme was transfected and confirmed in cells (SK-LU I) that have been known that there is no target, it was expectedly confirmed that the luciferase is leakily expressed in the absence of the target (see FIG. 11).

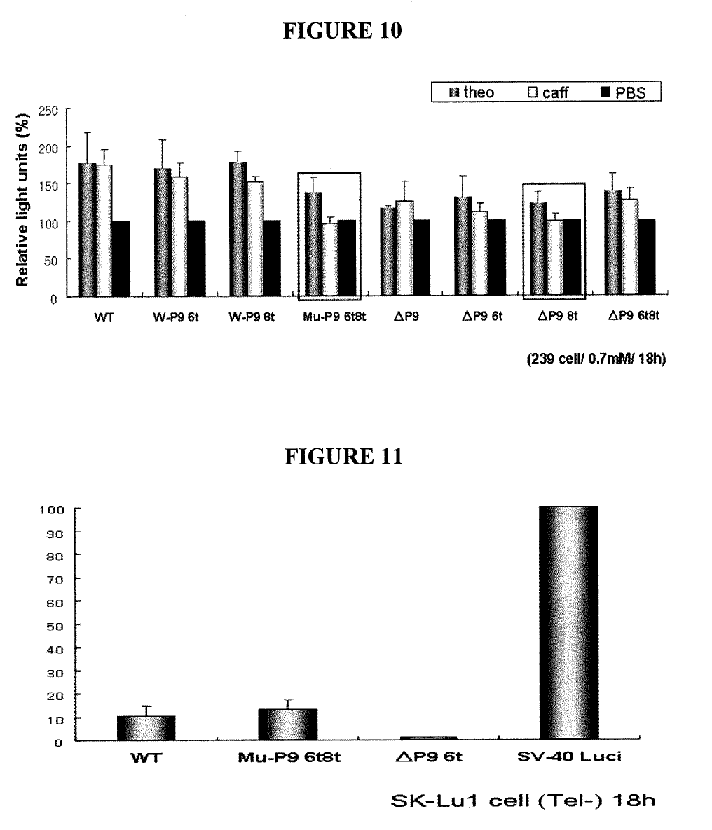

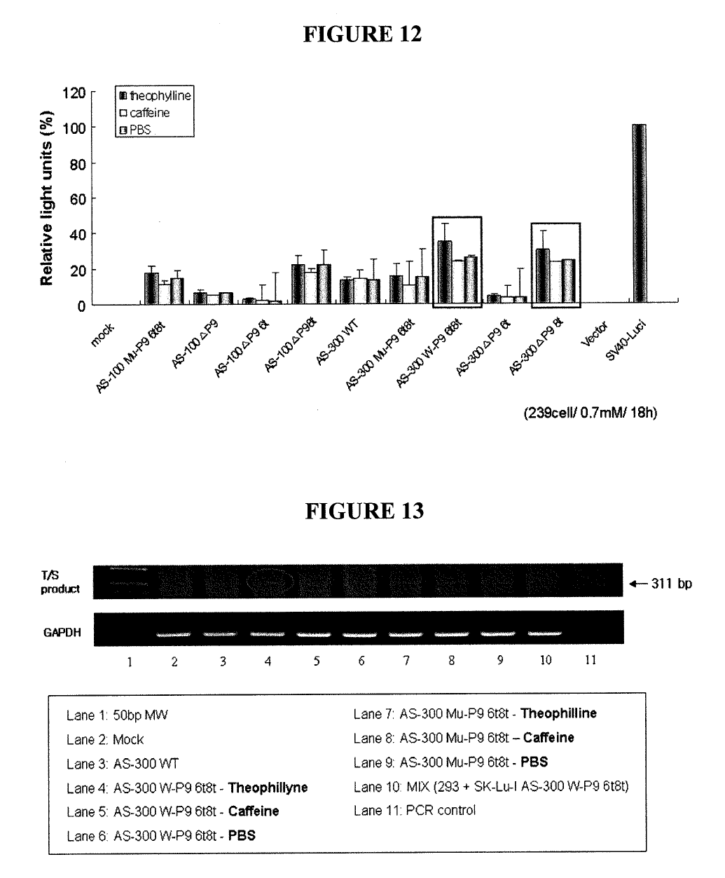

A dose of anti-sense RNA was increased to supplement this background. In expectation that the increase in the anti-sense RNA reduce the non-specific expression and further increase the efficiency, anti-sense RNA of the ribozyme was increased in dosage from 100 to 300, and the expression of luciferase was confirmed in the cells. As a result, it was revealed that an expression rate of luciferase is increased overall. As a consequence, it might be observed that the luciferase activity is effectively induced in hTERT+ cells in a theophylline-dependent manner in the case of AS-100 Mu-P9 6t8t, AS-300 W-P9 6t8t and AS-300 ΔP9 8t (see FIGS. 10 and 12). The total RNA of the cells was isolated to verify the trans-splicing in the cells, and a trans-splicing product was confirmed at the RNA level. As a result, it was confirmed that bands of the trans-splicing products are observed in the AS300 WT in a mock condition and the AS300 W-P9 6T8T at the presence of theophylline (see FIG. 13).

The 3′ exon was changed by substituting the luciferase with herpes simplex virus thymidine kinase (HSV-TK). That is to say, an allosteric trans-splicing group I ribozyme, which specifically targets hTERT RNA and has an HSV-TK apoptosis gene at 3′ exon, was prepared, and an expression vector (pAvQ-Theo-Rib21AS-TK) encoding the ribozyme was prepared. Then, the prepared expression vector was transfected into adenovirus, which was used later in experiments.

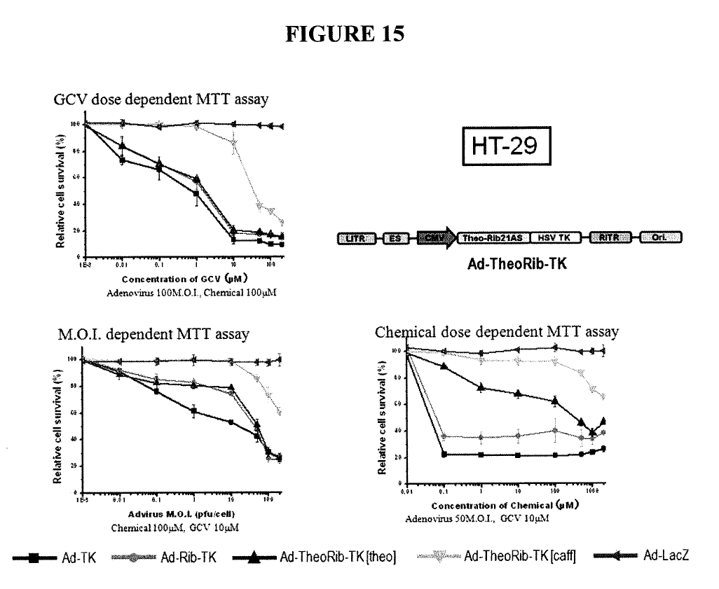

hTERT positive cell lines (HT-29, HepG2 and Capan-1) and a negative cell line (IMR90) were treated with a variety of adenovirus, then also treated with ganciclovir (GCV), theophylline and caffeine for 5 days, respectively, to observe apoptosis using an MTT assay. In this case, Ad-TK (an adenoviral vector expressing an HSVtk gene under the control of a CMV promoter) was used as a positive control, and Ad-Rib-TK (an adenoviral vector which is specific to hTERT and tagged with HSVtk) was used as a positive control in the hTERT+ cells. Also, Ad-LacZ (an adenoviral vector expressing a LacZ gene under the control of a CMV promoter) was used as a negative control. As a result, it was revealed that the adenoviral vectors, Ad-TK and Ad-Rib-TK, in the hTERT+ cell lines died regardless of the presence of regulator compounds when the hTERT+ cell lines were treated with GCV, but the Ad-TheoRib-TK was specifically apoptosized only at the presence of theophylline (see FIGS. 15 to 17). Also it was confirmed that the negative control, Ad-LacZ, was not apoptosized in every cases.

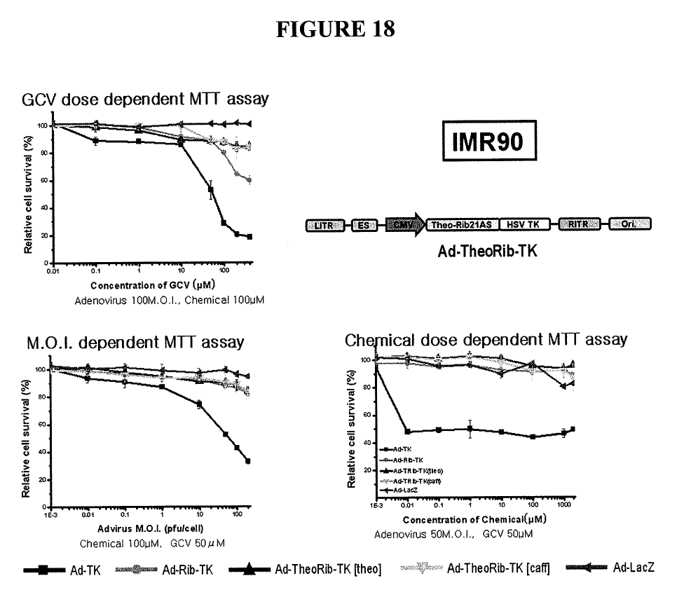

An hTERT− cell line, IMR90, was tested to determine whether the above-mentioned specific apoptosis is controlled by target RNA. As a result, the Ad-TK was apoptosized but the Ad-Rib-TK, Ad-TheoRib-TK and Ad-LacZ were not apoptosized when the hTERT− cell lines were treated with GCV. Therefore, it was confirmed that the apoptosis was controlled by the hTERT target RNA (see FIG. 18).

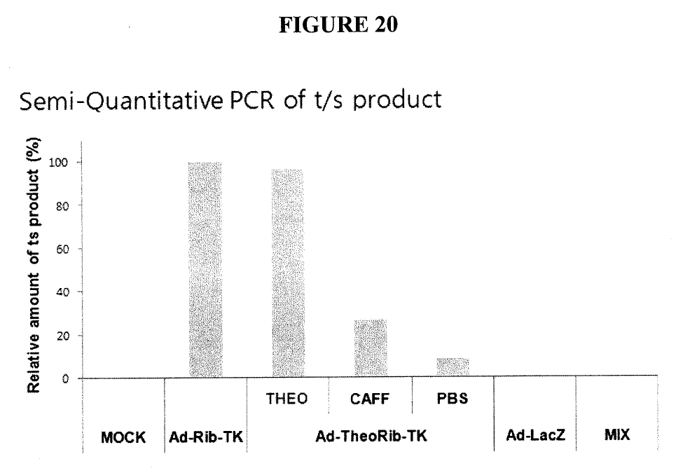

hTERT+ cells, HT-29, which contains 100 M.O.I adenovirus whose apoptosis was the most highly induced in the MTT assay, was treated 100 μM of a chemical to obtain the total RNA, and a small amount of the resulting total RNA were subject to a real-time PCR. As a result, it was confirmed that a trans-splicing product was observed only in the Ad-Theo-CRT-engrafted HT-29 cells in the presence of theophylline, and expressed at a substantially similar concentration, compared to when the hTERT+ cells were transfected with the Ad-Rib-TK. It was revealed that a trans-splicing product was produced at a significant concentration when the hTERT+ cells were treated with caffeine, but its concentration was decreased by approximately 78%, compared to when the hTERT+ cells were treated with theophylline (see FIG. 20). This indicates that the caffeine has 1000 times lower affinity to the theophylline aptamer than the theophylline, but has a somewhat significant affinity to the theophylline aptamer. From these results, it was seen that the induction of the target-specific apoptosis caused by the allosteric ribozyme according to one exemplary embodiment of the present invention was dependent on the theophylline in the cells, and initiated by the target RNA-specific trans-splicing reaction.

### EXAMPLES

Hereinafter, exemplary embodiments of the present invention are described in more detail with reference to the accompanying drawings, but the present invention is not particularly limited thereto.

### Reference Example 1

### Preparation of Substrate (hTERT) RNA

To prepare target RNA, a pCl-neo vector (exon 1-2) containing a −1st to +218th DNA sequence of the hTERT was PCR-amplified with a primer (5′-GGGGAATTCTAATACGACTCACTATAGGGCAGGCAGCGCTGCGTCCT-3′) set forth in SEQ ID NO: 9 and a primer (5′-CGGGATCCCTGGCGGAAGGAGGGGGCGGCGGG-3′) set forth in SEQ ID NO: 10, thus to prepare a DNA fragment encoding hTERT RNA. The DNA fragment thus prepared was transcribed in vitro into RNA. A DNA template (3 μg), a 10× transcription buffer, 10 mM DTT (Sigma), 0.5 mM ATP, GTP, CTP and UTP (Roche), an 80U RNase inhibitor (Kosco), a 200U T7 RNA polymerase (Ambion) were added, and DEPC-H₂O was added to a final volume of 100 μl, and then mixed. Then, the resulting mixture was reacted at 37° C. for 3 hours, and further treated with 5U DNase I (Promega) at 37° C. for 30 minutes to completely remove the DNA template. RNA was purified through the phenol extraction (pH 7.0) and ethanol precipitation, and separated on 6% denaturing polyacrylamide gel to elute an RNA band. Then, the RNA band was purified and dissolved in a TE buffer (10 mM Tris-HCl, pH 7.5, and 1 mM EDTA).

### Reference Example 2

### Cloning of Theophylline-Dependent hTERT Targeting Trans-Splicing (T/S) Aptazyme

As a basic trans-splicing ribozyme backbone used to develop an allosteric ribozyme, group I intron ribozyme, which specifically recognizes a +21 nt site of hTERT and has P1, P10 and extended IGS to which 300 nt anti-sense sequence against target RNA is annealed, was used (Kwon, B. S., Jung, H. S., Song, M. S., Cho, K. S., Kim, S. C., Kimm, K., Jeong, J. S., Kim, I. H., and Lee, S. W. 2005, Specific regression of human cancer cells by ribozyme-mediated targeted replacement of tumor-specific transcript. Mol. Ther. 12: 824-834; Hong, S. H., Jeong, J. S., Lee, Y. J., Jung, H. I., Cho, K. S., Kim, C. M., Kwon, B. S., Sullenger, B. A., Lee, S. W.*, and Kim, I. H.* 2008, In vivo reprogramming of hTERT by trans-splicing ribozyme to target tumor cells. Mol. Ther. 16: 74-80).

A theophylline aptamer was cloned into either or both of P6 and P8 domains of the hTERT targeting ribozyme by means of a communication module. Also in the case of the ΔP9 ribozyme where a P9 domain is deleted from the ribozyme, P6 and P8 domains of the hTERT targeting ribozyme were modified as the same manner as described above. By using a primer set forth in SEQ ID NO: 11 (5′-GGGGAATTCTAATACGACTCACTATAGGCAGGAAAAGTTATCAGGCA-3′) contained IGS that can target hTERT from the self-splicing ribozyme to which the theophylline aptamer is annealed via a communication module, and a primer set forth in SEQ ID NO: 12 (5′-CGAGTACTCCAAAACTAATCAA-3′) that can amplify a gene right upstream of 3′ exon of the ribozyme, a gene of the hTERT targeting trans-splicing ribozyme to which the theophylline aptamer is annealed was amplified, cleaved with restriction enzymes Hind III and Nru I, and then cloned into a SEAP promoter vector. A luciferase gene was PCR-amplified with a primer set forth in SEQ ID NO: 13 (5′-CGATGATCACGAAGACGC-3′) and a primer set forth in SEQ ID NO: 14 (5′-AAGGAAAAAAGCGGCCGCTTATTACAATTTGGACTTT-3′), cleaved with restriction enzymes Nru I and Xba I, and then cloned into a 3′ end of the ribozyme. However, a construct whose P9 domain is modified into an unexpected sequence was obtained in the cloning procedure using PCR. In addition to the construct, two wild constructs (i.e. wild P9 6t and wild P9 8t), 3 deleted constructs (i.e. ΔP9 6t, ΔP9 8t and ΔP9 6t8t) and a mutant construct (Mu P9 6t8t) were prepared, and an aptamer-free wild P9 and 8 constructs containing ΔP9 were prepared as the control.

A DND sequence of the prepared theophylline-dependent hTERT targeting T/S aptazyme was amplified in a total 10 μl of a reaction mixture including 3 μl of a terminator ready reaction mixture (PE applied Biosystems), 100 ng of quantified DNA, and 3.2 pmol of a primer set forth in SEQ ID NO: 15 (5′-CGGGATCCCTGGCGGAAGGAGGGGGCGGCGGG-3′) through 25 cycles (96° C.—10 sec, 50° C.—5 sec, and 60° C.—4 sec.). In order to purify the amplified gene of the theophylline-dependent hTERT targeting T/S aptazyme, 40 μl of dH2O was then added, and 3M NAACP ( 1/10 volume) and 100% EtOH (2 volumes) were added to the amplified reaction mixture. The resulting reaction mixture was vortexed, centrifuged at 4° C. for 30 minutes at a rotary speed of 13,000 rpm to obtain a DNA pellet. The DNA pellet was washed with 70% EtOH (400 μl), and dried in a speed-vacuum dryer to remove EtOH. The dried DNA pellet was dissolved in 15 μl of a template suppression reagent. Subsequently, the resulting DNA solution was vortexed and spun down, and transferred to a sequencing tube, and sequenced in an automatic sequence analyzer (ABI 310 Genetic Analyzer).

### Reference Example 3

### Preparation of Theophylline-Dependent hTERT Targeting T/S Aptazyme RNA

The DNA sequence of the theophylline-dependent hTERT targeting T/S aptazyme prepared in Reference example 2 was PCR-amplified with a primer set forth in SEQ ID NO: 16 (5′-GGGGAATTCTAATACGACTCACTATAGGCAGGAAAAGTTATCAGGCA-3′) including a T7 polymerase promoter and a primer set forth in SEQ ID NO: 17(5′-CCCAAGCTTGCGCAACTGCAACTCCGATAA-3′) that is annealed with the midway site of the 3′ exon of the ribozyme. In this case, an increased amount of a DNA template (3 μg) and NTP (1.5 mM) was used to prevent a self-splicing reaction as much as possible. Then, a 1× splicing buffer (40 mM Tris-HCl, pH 7.5, 5 mM MgCl₂, 10 mM DTT and 4 mM spermidine), 0.5 mM ATP, GTP, CTP, UTP (Roche), an 80U RNase inhibitor (Kosco), a 200U T7 RNA polymerase(Ambion) were added, and DEPC-H₂O was added to a final volume of 100 μl and then mixed. Then, the resulting mixture was transcribed at 37° C. for 3 hours, and further treated with 5U DNase I (Promega) at 37° C. for 30 minutes to completely remove the DNA template. RNA was purified through the phenol extraction (pH 7.0) and ethanol precipitation, and separated on 4% denaturing polyacrylamide gel to elute an RNA band. Then, the RNA band was purified and dissolved in a TE buffer (10 mM Tris-HCl, pH 7.5, and 1 mM EDTA).

### Reference Example 4

### In Vitro Trans-Splicing Reaction

The ribozyme (50 nM) and the substrate RNA, hTERT RNA, (10 nM) were reacted at 37° C. for 3 hours in the presence theophylline (500 μM) or caffeine (500 μM) having one different residue from the theophylline, or an equivalent volume of dH₂O under the splicing condition (50 mM HEPES, pH 7.0/150 mM NaCl/5 mM MgCl₂/100 μM guanosine), and the resulting reaction product was analyzed in a RT-PCR reaction. In this case, a luciferase recognition site (5′-CCCAAGCTTGCGCAACTGCAACTCCGATAA-3′, SEQ ID NO: 18) was used as the primer for reverse transcription (RT), and a site (5′-GGAATTCGCAGCGCTGCGTCCTGCT-3′, SEQ ID NO: 19) that recognizes a 5′ end of the hTERT RNA and a site (5′-CCCAAGCTTTCACTGCATACGACGATT-3′, SEQ ID NO: 20) that recognizes a luciferase gene were used as the 5′ and 3′ primers for polymerase chain reaction (PCR), respectively.

### Reference Example 5

### Semi-Quantitative PCR

After the in vitro trans-splicing reaction, the trans-splicing product was subject to a real-time PCR, followed by a semi-quantitative PCR. Each DNA sample was tested in triplet to calculate an average value and determine its melting point, and the DNA samples were observed on agarose gel. In this case, the DNA samples were detected with SYBR Green, and a standard control quantified from the RT reaction was used to semi-quantitatively compare to the DNA samples. For the purpose of correction, an equivalent amount of any RNA (ras RNA) was added to each sample during the RT reaction, and the RT primer was designed so that the trans-splicing product and an internal control, ras RNA, could be reversely transcribed by one primer. Here, a primer set forth in SEQ ID NO: 21 (5′-GCCCAACACCGGCATAAAGTTACATAATTACACACTT-3′) was prepared as the RT primer. Therefore, concentrations of the reversely transcribed samples were corrected with a concentration of the ras cDNA for the quantitative comparison of the reversely transcribed samples.

The PCR reaction was carried out under the PCR conditions: preheating at 96° C. for 10 minutes, denaturation at 96° C. for 5 minutes, annealing at 60° C. for 15 seconds, and extension at 72° C. for 30 seconds. In this case, an hTERT recognition site (5′-CCCGAATTCTGCGTCCTGCTCGA, SEQ ID NO: 22) was used as the 5′ primer, and a luciferase recognition site (5′-CCCAAGCTTTCACTGCATACACGATT, SEQ ID NO: 23) was used as the 3′ primer. As the internal control, PCR primers of the ras cDNA were used, as follows: 5′ primer (5′-ATGACTGAATATAAACTT, SEQ ID NO: 24) and 3′ primer (5′-CCCAAGCTTTACATAATTACACACTT, SEQ ID NO: 25).

### Reference Example 6

### Preparation of Specific T/S Aptazyme with Improved Specificity

A complementary 100 nt anti-sense strand toward a 3′ end of the hTRET sequence that is recognized on the hTRET sequence by an intergenic spacer (IGS) was PCR-amplified with a primer set forth in SEQ ID NO: 26 (5′-AATTCAAGCTTCGTTTTGCGGCAGCAGGAAAAGTTATCAGGCATG-3′) and a primer set forth in SEQ ID NO: 27 (5′-CCTGATAACTTTTCCTGCCGCAAAACGAAGCTTG-3′), and a 300 nt anti-sense strand was PCR-amplified with a primer set forth in SEQ ID NO: 28(5′-GGGAAGCTTGGGAAGCCCTGGCCC-3′) and a primer set forth in SEQ ID NO: 29(5′-GGGAAGCTTAAGGCCAGCACGTTCTT-3′). Then, the amplified anti-sense strands were cloned into a Hind III restriction site upstream of the previously prepared ribozyme construct.

### Reference Example 7

### Cell Culture

An hTERT positive cell line was cultured at 37° C. in a 5% CO₂ incubator with reference to 293 (human kidney/normal), HT-29 (colon/colorectal adenocarcinoma), Capan-1 (pancreas/adenocarcinoma) and HepG2 (liver/hepatocellular carcinoma), and an hTERT negative cell line was cultured at 37° C. in a 5% CO₂ incubator with reference to IMR-90 (lung/fibroblast/normal) and SK-LU1(lung/adenocarcinoma) ATCC.

### Reference Example 8

### Verification of Specificity and Efficiency of Trans-Splicing Aptazyme in Cell Lines

Test for Optimum Concentration of Theophylline

293 cells were seeded in a 35 mm dish at a concentration of 3×10⁵, and grown to approximately 80% confluence. In this case, the grown 293 cells were transfected with 1 μg of the Mu P9 6t8t construct using LipofectAMINE (Invitrogen). The transfected 293 cells were cultured for 18 hours at increasing concentrations (0.1 mM, 0.3 mM, 0.5 mM, 0.7 mM, and 1 mM) of theophylline or caffeine, respectively, and then subjected to a luciferase assay. An equivalent volume of PBS was used as the control.

2) Dual Luciferase Assay

Media of the transfected cells were removed from the 35 mm dishes, and washed with 1×PBS. Next, 200 p. 1 of a 1× passive lysis buffer was added to each transfected cells, and the transfected cells were lysed at a room temperature for 15 minutes to obtain a cell lysate. The cell lysate was centrifuged for 1 minute in a rotary speed of 13,000 rpm, and the resulting supernatants were transferred respectively to new tubes. 100 μl of LARII (Luciferase assay reagent II) was put into a luminometer tube, and 20 μl of the cell lysate was also added and mixed. Then, the resulting mixture was read using a luminometer. 100 μl of Stop & Glo reagent mix (Stop & Glo 20 μl+Stop & Glo buffer 1 ml) was added again to the luminometer tube, and mixed. Then, the resulting mixture was also read using a luminometer (TD+20/20). A delay time was set to 3 seconds, an integration time was set to 12 seconds, and the sensitivity was set to 45% which was suitable for each cell to be measured.

For the transfection, the cells were treated with the theophylline and caffeine dissolved in PBS. Also, when the used MEM medium was exchanged with a new MEM medium after the transfection of the cells, the cell cultures were treated with each chemical, incubated for 18 hours, and then subjected to a luciferase assay.

3) Intracellular Trans-Splicing Reaction

293 cells were transiently transfected with 1 μg of a ribozyme vector using 4 μl of lipofectamine. 5 hours after the transfection, the used medium was exchanged with a fresh medium supplemented with 0.7 mM theophylline or caffeine, and kept for 18 hours to obtain a cell lysate. Then, the total RNA was purified from the cell lysate. In this case, the RNA was extracted using a guanosine isocyanate cell lysate solution supplemented with 20 mM EDTA so as to minimize the possibility of in vitro trans-splicing reaction. The extracted RNA was reversely transcribed with a primer (5′-CCCAAGCTTGCGCAACTGCAACTCCGATAA, SEQ ID NO: 30) that recognizes a luciferase gene, thus to obtain cDNA. The cDNA was PCR-amplified with a nested luciferase primer (5′-CCCAAGCTTGCCCAACACCGGCATAAAG, SEQ ID NO: 31) as the 3′ primer and a recognition site (5′-AGCGCTGCGTCCTGCT, SEQ ID NO: 32) that recognizes a 5′ end of the hTERT as the 5′ primer. A 40 cycle PCR reaction was carried out under the PCR conditions of: preheating at 96° C. for 10 minutes, denaturation at 96° C. for 5 minutes, annealing at 58° C. for 30 seconds, extension at 72° C. for 20 seconds. In this case, the RNA extracted as the reaction control for the reaction product was reversely transcribed with oligo dT, and the resulting cDNA was amplified with a GAPDH 5′ primer (5′-TGACATCAAGAAGGTGGTGA, SEQ ID NO: 33) and a GAPDH 3′ primer (5′-TCCACCACCCTGTTGCTGTA, SEQ ID NO: 34) to observe an expression level of GAPDH RNA, which was used as an internal control.

4) Preparation of Adenovirus that Expresses Theophylline-Dependent hTERT Targeting T/S Aptazyme

A pAvQ shuttle vector was cleaved with restriction enzymes BamHI and BstBI, and DNA fragments, WT P9-TK and AS300 W-P9 6T8T-TK, were cloned into the pAvQ shuttle vector to prepare a vector that expresses ribozyme under the control of a CMV promoter in mammalian cells. The prepared vector was linearized with a restriction enzyme Pme I, and co-transfected into BJ5183 bacteria together with a type 5 adenovirus genome DNA plasmid, ΔE1/E3 pAdenovector (Qbiogene), using an electroporation method. A recombinant adenoviral vector construct obtained in bacteria cells through homologous recombination was separated, purified, and checked by miniprep. Then, the recombinant adenoviral vector construct was linearized with a restriction enzyme Pac I, and transfected into a packaging cell line, 293 cells. Plaque clones formed through viral proliferation were obtained, and cell debris was removed to obtain a virus supernatant. The infected 293 cells were infected with the virus supernatant to verify whether hemolysis of the cells occurred.

The adenoviral vectors expressing AS300 WT P9-TK (original T/S ribozyme) and AS300 W-P9 6T8T-TK (allosteric T/S ribozyme) under the control of the CMV promoter were named Ad-Rib-TK and Ad-TheoRib-TK, respectively.

In order to determine whether the recombinant adenoviruses (Ad-Rib-TK and Ad-TheoRib-TK) had been prepared successively, the 293 cells were infected with the supernatant obtained from the recombinant virus genome DNA-transfected 293 cells, and the recombinant adenoviruses were verified through the cytopathic effect (CPE). Also, the recombinant adenoviruses were verified by obtaining DNA from a virus supernatant, which was obtained from the plaque clone inducing the cell lysis, and undergoing a PCR experiment (TK and virus ITR sites) on the DNA.

RNA was extracted from a lysate of the virus-infected cells, and a RT-PCR on the RNA (TK RNA) was carried out to verify whether the recombinant virus construct was prepared successively and the transgenes from this virus were expressed. The 293 cells were infected several times with the recombinant viruses, obtained from the supernatant of the 293 cells infected with each recombinant adenovirus clone, thereby amplifying the recombinant viruses. Then, the recombinant adenoviral vector was separated and purified using Vivapure® AdenoPACK™. The resulting recombinant virus was diluted continuously, and then subjected to a TCID50 assay to determine a PFU titer of the purified virus vector.

5) MTT Assay

Cells were seeded in a 96 well plate (TPP) for 1 day, and then infected with Ad-TK (an adenoviral vector expressing a TK gene under the control of a CMV promoter), Ad-Rib-TK, Ad-TheoRib-TK, and Ad-LacZ (an adenoviral vector expressing a LacZ gene under the control of a CMV promoter) adenoviruses, respectively. The used media supplemented with GCV and a chemical (theophylline or caffeine) were exchanged with the same new media from every second to fifth day from the day after the virus infection. The HT-29 strain was seeded at a cell number of 3×10³/well, the HepG2 strain was seeded at a cell number of 3×10³/well, the Capan-1 strain was seeded at a cell number of 5×10³/well, and the IMR90 strain was seeded at a cell number of 5×10³/well. 5 days after the seeding of the strains, CellTiter 96® AQueous ONE Solution Cell Proliferation Assay (Promega) was added to each cell medium so that it amounted to 20% of the total medium, and the 96 wells were treated with 100 μl of resulting cell medium per well, and measured at a wavelength of 490 nm, using a Microplate reader model 550 (BioRad), to observe cell viability of the cells.

6) Semi-Quantitative PCR

A 35 mm dish was infected with adenovirus, and the used media supplemented with a chemical (theophylline or caffeine) were exchanged with the same new media after 24 hours of the infection. After 24 hours of the medium exchange, RNA was purified from the cultured cells using a TriZol reaction reagent (Invitrogen), and reversely transcribed, and subjected to a semi-quantitative PCR on the trans-splicing product using a real-time PCR. A concentration of the T/S PCR product was corrected with the concentration of the GAPDH PCR product.

RNA was reversely transcribed with oligos (dT), and the resulting cDNA was amplified with a TK primer (5′-CCCATGCACGTCTTTATCCTGGAT-3′, SEQ ID NO: 35) as the 3′ primer and a site (5′-GGAATTCGCAGCGCTGCGTCCTGCT-3′, SEQ ID NO: 36) that recognizes a 5′ end of the hTERT as the 5′ primer, by using a real-time PCR. The RNA extracted as the reaction control for the reaction product was reversely transcribed with oligo dT, and the resulting cDNA was amplified with a GAPDH 5′ primer (5′-TGACATCAAGAAGGTGGTGA, SEQ ID NO: 37) and a GAPDH 3′ primer (5′-TCCACCACCCTGTTGCTGTA, SEQ ID NO: 38) to observe the expression level of GAPDH RNA, which was used as an internal control.

### Example 1

### Preparation of Trans-Splicing Ribozyme that has a Theophylline Aptamer Attached Thereto and Specifically Targets hTERT RNA

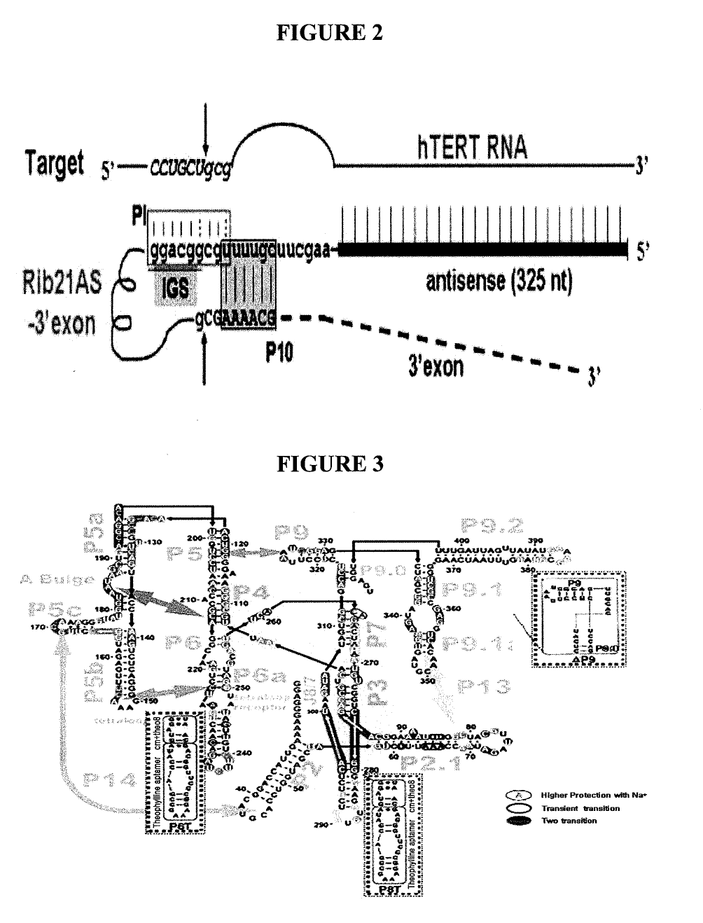

As a basic trans-splicing ribozyme backbone used to develop allosteric ribozymes, group I intron ribozyme, which specifically recognizes a +21 nt site of hTERT and has P1, P10 and extended IGS to which 300 nt anti-sense sequence against target RNA is annealed, was used (FIG. 2). It was observed that this ribozyme induces the hTERT-expressing cancer cell-specific apoptosis by specifically expressing hTERT RNA in cell and animal models (Mol. Ther. 2005; 12:824, Mol. Ther. 2008; 16:74).

In order to prepare theophylline-dependent allosteric ribozyme, a theophylline RNA aptamer (Science 1994; 263:1425) used as a receptor domain of theophylline was simultaneously attached to either or both of P6 or/and P8 domains, which play an important role in RNA folding for the catalytic functions of the hTERT-specific T/S ribozyme developed by the research team of this application. Also, a T/S ribozyme was prepared by binding a theophylline aptamer to a P6, P8, or P6+P8 domain of the ribozyme whose P9 domain was substituted with a minimized ΔP9 domain or modified. FIG. 3 shows a structure and an RNA sequence of a group I intron which is homologous to the trans-splicing ribozyme, a theophylline aptamer, and a communication module structure where the theophylline aptamer is annealed to ribozyme, etc. (Nucleic Acid Res. 2002; 30:4599).

The prepared trans-splicing ribozyme constructs were listed, as follows.

hTERT-specific trans-splicing ribozyme (WT)

Ribozyme (W-P9 6t, WT-P9 8t) where an aptamer is attached to a P6 or P8 domain of WT

Ribozyme (Mu-P9 6t8t) where an aptamer is attached to P6 and P8 domains of a mutant P9

Ribozyme (ΔP9) where a P9 domain of WT ribozyme is substituted with ΔP9

Ribozyme (ΔP9 6t, ΔP9 8t, ΔP9 6t8t) where an aptamer is attached to P6, P8, P6+P8 domains of the ΔP9 ribozyme

WT ribozyme (AS-300 WT) to which an anti-sense 300 nt sequence against hTERT is attached

WT ribozyme (IGS W-P9 6t8t) having P1 and P10 helixes and containing an aptamer attached to a P6+P8 domain

WT ribozyme (AS-300 W-P9 6t8t) having an anti-sense 300 nt sequence attached thereto and containing an aptamer attached to a P6+P8 domain

Mu-P9 ribozyme (AS-300 Mu-P9 6t8t) having an anti-sense 300 nt sequence attached thereto and containing an aptamer attached to a P6+P8 domain

A structure of the mutant P9 was spontaneously prepared in a PCR procedure of preparing a ribozyme vector, and it was revealed that the structure of the mutant P9 did not affect the activities of ribozyme when it was subject to an in vitro trans-splicing reaction with target RNA (hTERT RNA). Therefore, as one of the candidates to prepare allosteric ribozyme according to the present invention, a ribozyme construct based on the mutant P9 was also prepared, and its functions were determined. FIG. 4 shows a wild-type P9 sequence and a mutant P9 (Mu-P9) sequence. The other sequence regions were represented by bold and underlined letters.

### Example 2

### Quantitative Analysis of Ribozymes Having an Ability to Substitute Theophylline-Dependent RNA

The ribozyme and the hTERT RNA as the substrate RNA, as thus prepared, were reacted at 37° C. for 3 hours under the splicing condition, and the resulting splicing product was analyzed through a RT-PCR reaction. In the splicing reaction, water, or 0.5 mM caffeine (a theophylline structure analogue, a negative control for the specificity of allosteric effects), or 0.5 mM theophylline were reacted together to observe whether the trans-splicing reaction was allosterically turned on in a theophylline-specific manner. FIG. 5 shows the electrophoretic results of the RT-PCR product.

Referring to FIG. 5, it was revealed that the WT and ΔP9 ribozymes always induced the trans-splicing reaction regardless of caffeine, theophylline and water as it was expected, and the W-P9 6t also induced the trans-splicing reaction regardless of the compounds. Also, it was revealed that the W-P9 8t did not induce the trans-splicing reaction in a theophylline-specific manner, and the trans-splicing reactions might be ineffectively induced in the case of the ΔP9 8t and ΔP9 6t8t. Meanwhile, it was revealed that a trans-splicing product with 319 by size was produced in vitro in a theophylline-specific manner in the case of the Mu-P9 6t8t and ΔP9 6t ribozymes. Therefore, it was revealed that the P6 or P8 domains of the group I intron was a main domain that can allosterically control the ribozyme activities in a theophylline-dependent manner, depending on the P9 sequence or structural characteristics of the ribozyme.

In order to compare and analyze a control degree of the induction of the theophylline-dependent trans-splicing reaction by the allosteric ribozyme, real-time PCR analysis (Analysis apparatus: Corbett Research RG 6) for the trans-splicing product was carried out. The Mu-P9 6t8t ribozyme whose enzyme activity is controlled through the trans-splicing reaction in a theophylline-dependent manner or the WT ribozyme having structural enzyme activities regardless of the small molecules compounds was spliced with hTERT RNA, followed by subjecting to a RT reaction. For the quantitative comparison of the reversely transcribed samples, concentrations of the reversely transcribed samples were corrected using the concentration of ras cDNA. The results are shown in FIG. 6. Referring to FIG. 6, it was revealed that an equivalent concentration of the trans-splicing product was produced in a splicing buffer regardless of the presence of water, theophylline and caffeine in the case of the WT ribozyme. From the real-time quantitative analysis of the reaction product, it was also revealed that a theophylline-dependent trans-splicing reaction did not occur in the ΔP9 6t ribozyme. However, it was revealed that, when concentrations of the trans-splicing products in the respective RT samples were corrected with the internal control, the trans-splicing product was produced at a 4.3 times higher concentration in the presence of theophylline than in the presence of caffeine, and produced at a 12.16 times higher concentration than when in the presence of an equivalent volume of dH₂O, in the case of the Mu-P9 6t8t ribozyme whose activity is controlled in vitro in a theophylline-dependent manner as described in the previous experiment. Therefore, it was revealed that the Mu-P9 6t8t ribozyme was an allosteric ribozyme whose trans-splicing reaction may be effectively controlled in vitro in a theophylline-dependent manner.

Since the intergenic spacers (IGS) of the analyzed ribozymes have only a 6 nt sequence, ribozymes having an extended IGS group should be used to perform a target RNA-specific trans-splicing reaction in cells (Nat. Biotechnol. 1996; 15:902, J Mol. Biol. 1999; 185:1935, Mol. Ther. 2003; 7:386, Mol. Ther. 2004; 10:365; Mol. Ther. 2005; 12:824). The ribozymes having an extended IGS were prepared by the in vitro transcription, and then subject to an in vitro trans-splicing reaction with hTERT RNA. In this case, it was also observed whether the trans-splicing reaction was carried out in a theophylline-dependent manner. The prepared ribozymes include WT ribozyme (AS-300 WT) to which an anti-sense 300 nt sequence against hTERT is attached; WT ribozyme (IGS W-P9 6t8t) having P1 and P10 helixes and containing an aptamer attached to a P6+P8 domain; WT ribozyme (AS-300 W-P9 6t8t) having an anti-sense 300 nt sequence attached thereto and containing an aptamer attached to a P6+P8 domain; and Mu-P9 ribozyme (AS-300 Mu-P9 6t8t) having an anti-sense 300 nt sequence attached thereto and containing an aptamer attached to a P6+P8 domain, and their trans-splicing reaction results (RT-PCR products of the trans-splicing products) are shown in FIG. 7.

In FIG. 7, it was revealed that the AS-300 WT induced the splicing reaction regardless of the theophylline, as expected. Also, it was revealed that the AS300 W-P9 6t8t induced the in vitro splicing reaction regardless of theophylline, but the AS300 Mu-P9 6t8t did not easily induce the splicing reaction unlike the AS300-free ribozyme. Meanwhile, it was revealed that the IGS W-P9 6t8t that is free from an anti-sense sequence and has P1 and P10 helixes induced the trans-splicing reaction only at the presence of theophylline. These results indicate that, although the ribozyme having a 6 nt IGS sequence and the ribozyme having an extended IGS sequence have the same basic backbone structure, they might have a structural difference where they show their different activities according to the presence of the anti-sense sequence. Therefore, this indicates that the splicing activities of the ribozymes having an extended IGS sequence should be observed in vitro and/or in vivo.

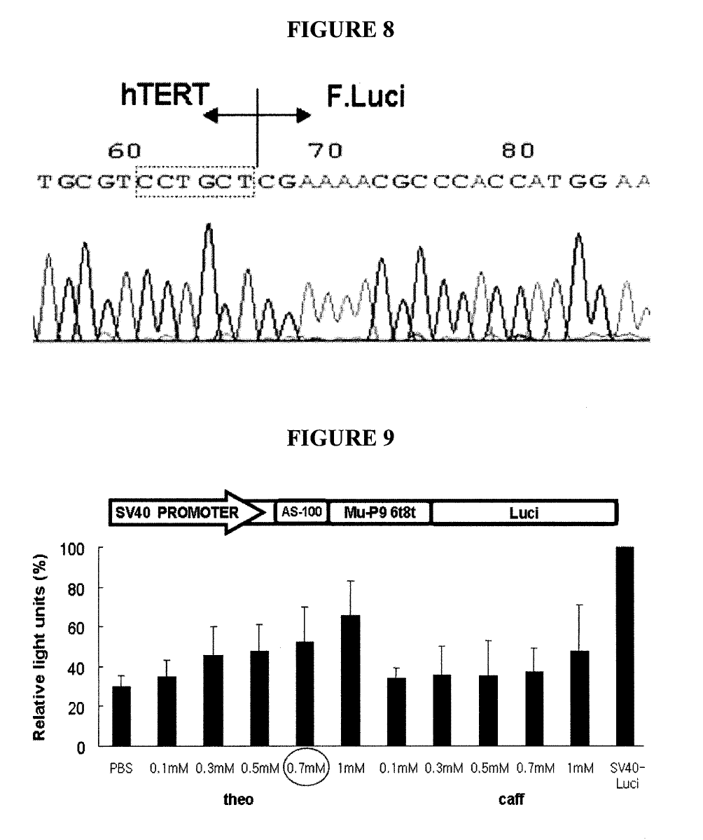

From the above results, it was seen that the trans-splicing activity is allosterically controlled in vitro in a theophylline-dependent manner in some ribozymes to which a theophylline aptamer is attached. In order to verify whether the trans-splicing product is produced in the exact trans-splicing reaction, the prepared trans-splicing RT-PCR product was cloned into a pUC19 vector, and sequenced. As shown in FIG. 8, from the sequencing data of the trans-splicing reaction product, it was confirmed that firefly luciferase RNA attached to the 3′ exon of the ribozyme was exactly annealed to the downstream site of a +21st nt site of the hTERT RNA as the target RNA. This result means that the trans-splicing reaction of the allosteric trans-splicing ribozyme occurred very accurately.

### Example 3

### Preparation of Allosteric Trans-Splicing Ribozymes Having a Reporter Gene Attached to 3′ Exon

In order to prepare an expression vector of the theophylline-dependent allosteric ribozyme, a theophylline RNA aptamer (Science 1994; 263:1425) used as a receptor domain of theophylline was simultaneously attached to either or both of P6 or/and P8 domains, which play an important role in RNA folding for the catalytic functions of the hTERT-specific T/S ribozyme developed by the research team of this application (Nucleic Acid Res. 2002; 30:4599). Also, trans-splicing ribozymes were prepared by binding a theophylline aptamer to a P6, P8, or P6+P8 domain of the ribozyme whose P9 domain was substituted with a minimized ΔP9 domain or modified. As a transgene for inducing the expression of ribozyme, a firefly luciferase gene was inserted into a 3′ exon of the ribozyme, and a SV40 promoter system was used to facilitate intracellular expression of the ribozyme. The prepared trans-splicing ribozyme constructs are listed, as follows.

The construction of a vector was carried out by PCR-amplifying a sequence from a ribozyme region of the allosteric ribozyme constructs prepared for the in vitro splicing reaction to a 3′ end of the luciferase gene, inserting the amplified DNA between Hind III and Xba I restriction sites of a pSEAP vector (Clontech) containing a SV40 promoter, and inserting an anti-sense sequence against the hTERT RNA into a HindIII restriction site. In this case, the 5′ primer used to amplify the ribozyme contains P1 and P10 helixes and an IGS sequence that recognizes a +21st nt of the hTERT RNA (5′-GGGGAATTCTAATACGACTCACTATAGGCAGGAAAAGTTATCAGGCA-3′, SEQ ID NO: 39).

1). A vector containing a 100 nt anti-sense sequence against hTERT RNA;

An expression vector for hTERT-specific ribozyme (AS-100 WT)

An expression vector for ribozyme (AS-100 W-P9 6t, AS-100 WT-P9 8t) where an aptamer is attached to P6 and P8 domains of the WT ribozyme

An expression vector (pSEAP AS100 Mu-P9 6T8T-Luci, SEQ ID NO: 5) for ribozyme (AS-100 Mu-P9 6t8t) where an aptamer is attached to a P6+P8 domain of the mutant P9

An expression vector for ribozyme (AS-100 ΔP9 6t, AS-100 ΔP9 8t, AS-100 ΔP9 6t8t) where an aptamer is attached to P6, P8 and P6+P8 domains of the ΔP9 ribozyme

2). A vector containing a 300 nt anti-sense sequence against hTERT RNA;

An expression vector for hTERT-specific ribozyme (AS-300 WT)

An expression vector (pSEAP AS300 W-P9 6T8T-Luci, SEQ ID NO: 6) for ribozyme (AS-300 W-P9 6t8t) where an aptamer is attached to a P6+P8 domain of the WT ribozyme

An expression vector for ribozyme (AS-300 Mu-P9 6t8t) where an aptamer is attached to a P6+P8 domain of the mutant P9

An expression vector for ribozyme (AS-300 ΔP9 6t) where an aptamer is attached to a P6 domain of the ΔP9 ribozyme

An expression vector (pSEAP AS300 Delta P9 8T-Luci, SEQ ID NO: 4) for ribozyme (AS-300 ΔP9 8t) where an aptamer is attached to a P8 domain of the ΔP9 ribozyme

### Example 4

### Intracellular Observation of Specific Replacement of Compound-Dependent hTERT RNA of Ribozymes

Establishment of Induction Conditions of Theophylline-Dependent Trans-Splicing Transgene

First of all, the induction conditions of the allosteric ribozymes having a luciferase gene attached to 3′ exon as prepared above were established by determining at what intracellular theophylline concentration the expression of a transgene was the most allosterically induced.

293 cells were transiently transfected with the Mu-P9 6t8t ribozyme expression vector, which had induced the trans-splicing reaction in a theophylline-dependent manner, with lipofectamine through the in vitro trans-splicing reaction. In this case, in order to measure transfection efficiency and normalize the activity of an expressed product, the 293 cells were co-transfected with a vector that can express a renillar luciferase gene under the control of a CMV promoter. 4 hours after the transfection, the used medium was exchanged with a fresh medium. In this case, 0.1 mM, 0.3 mM, 0.5 mM, 0.7 mM and 1 mM of caffeine or theophylline was added to the fresh medium to verify what concentration of the caffeine or theophylline most induces the luciferase activity in the theophylline-dependent manner. 18 hours after the exchange with the fresh medium, a cell lysate was obtained, and then measured for firefly luciferase activity normalized to the renillar luciferase activity using a luminometer TD-20/20 (Turner Designs Instrument). In this case, the measured luciferase activity as shown in FIG. 9 as a relative value (%) to the concentration of the luciferase produced after the transfection of the vector (SV40-Luci), was shown to be able to express a luciferase gene under the control of the SV40 promoter.

Referring to FIG. 9, it was revealed that the theophylline-specific luciferase activity was most induced in the cells at the presence of 0.7 mM theophylline, compared to the presence of 0.7 mM caffeine. Therefore, the optimum theophylline concentration condition to induce the expression of the theophylline-dependent genes from various ribozyme expression vectors was fixed to 0.7 mM, and the following experiments were carried out under that concentration.

2) Induction of Expression of Theophylline-Dependent Transgene from Ribozyme Expression Vector Containing a 100 nt Anti-Sense Sequence

Induction of the intracellular transgene activity by the ribozymes, which contain a 100 nt anti-sense sequence against the hTERT RNA and has a theophylline aptamer attached thereto was also observed through the luciferase assay.

In this case, the measured luciferase activity was represented by a relative value (%) to a concentration of the luciferase observed from the PBS-treated cell lysate. The results are shown in FIG. 10.

Referring to FIG. 10, it was revealed that the AS-100 Mu-P9 6t8t ribozyme most induced the luciferase activity in a theophylline-specific manner, which accords with the in vitro data. However, it was revealed that the AS-100 ΔP9 8t ribozyme induced the expression of the transgene in a more theophylline-specific manner than the AS-100 ΔP9 6t ribozyme, which is different from the in vitro data. In this case, it is considered that the above results owed themselves to the structural changes in the ribozyme that may be caused by further adding a 100 nt anti-sense sequence to an upstream region of IGS, and also owing to the fact that the intracellular environments are surely different from the in vitro environments. Meanwhile, it might be considered that the WT, W-P9 6t, W-P9 8t and ΔP9, and ΔP9 6t8t ribozymes induce the transgene activity in the cells in a theophylline-specific manner.

3) Allosteric Ribozyme Activity in hTERT-Negative Cells

In order to determine, from the above results, whether the AS-100 Mu-P9 6t8t ribozyme, which was observed to allosterically induce the transgene activity in a theophylline-dependent manner in the cells, and the AS-100 ΔP9 6t ribozyme, which did not show an allosteric effect in the cells, induce the transgene activity in an hTERT target RNA-specific manner, the hTERT negative cells, SK-Lu-1 cells, were transiently transfected with each of the ribozyme vectors using DMRIE-C. 4 hours after the transfection, the used medium was exchanged with a fresh medium supplemented with theophylline. 18 hours after the exchange with the fresh medium, a cell lysate was obtained, and then measured for luciferase activity. In this case, the measured luciferase activity is shown in FIG. 11 as a relative value (%) to the expression level of the luciferase from the SV40-Luci vector.

Referring to FIG. 11, it was seen that, like the AS-100 WT ribozyme, both the AS-100 Mu-P9 6t8t and AS-100ΔP9 6t ribozymes suppressed the induction of the transgene expression regardless of the presence of theophylline when the target RNA was not present in the ribozymes. That is to say, it was revealed that theophylline-dependent allosteric trans-splicing ribozymes might induce the transgene expression in a target RNA-specific manner.

4) Induction of Expression of Theophylline-Dependent Transgene from Ribozyme Expression Vector Containing a 300 Nt Anti-Sense Sequence

In order to observe whether the induction of the allosteric transgene expression of the trans-splicing ribozyme may be enhanced with the increase in length of the anti-sense sequence, the ribozyme vectors containing a 300 nt anti-sense sequence against the hTERT RNA were prepared, and the induction of the theophylline dependent luciferase activity in the cells was compared and observed.

In this experiment, the AS-300 WT ribozyme was used as a theophylline-dependent control, and the hTERT positive cells, 293 cells, were co-transfected with each of the expression vectors for ribozyme (AS-300 Mu-P9 6t8t) whose Mu-P9 6t8t basic backbone contains a 300 nt anti-sense sequence, which induced the in vitro trans-splicing reaction in a theophylline-dependent manner and also induced the theophylline dependent transgene activity in the cells when the ribozyme contain an AS-100 sequence; ribozyme (AS-300 ΔP9 6t) whose ΔP9 6t basic backbone contains a 300 nt anti-sense sequence, which induced the in vitro trans-splicing reaction in a theophylline-dependent manner; ribozyme (AS-300 ΔP9 8t) whose ΔP9 8t basic backbone contains a 300 nt anti-sense sequence, which induced the theophylline dependent transgene activity in the cells when the ribozyme contained an AS-100 sequence; and ribozyme (AS-300 W-P9 6t8t) whose W-P9 6t8t basic backbone contains a 300 nt anti-sense sequence, which induced the in vitro trans-splicing reaction in a theophylline-dependent manner when the ribozyme contained an extended IGS sequence (P1+P10 helix). Then, the luciferase activities were measured, and the induction of the theophylline dependent gene activity was also compared and observed. In this case, the measured luciferase activity was shown in FIG. 12 as a relative value (%) to the concentration of the luciferase produced after the transfection of the vector (SV40-Luci) that can express a luciferase gene under the control of the SV40 promoter.

Referring to FIG. 12, it was revealed that the AS-300 WT ribozyme effectively induced the luciferase expression regardless of the presence of theophylline, as it was expected. Among the ribozymes containing a 300 nt anti-sense sequence, it was seen that the AS-300 Mu-P9 6t8t and AS-300 ΔP9 6t ribozymes did not induce the theophylline dependent transgene activity. Meanwhile, it was seen that the AS-300 W-P9 6t8t and AS-300 ΔP9 8t ribozymes effectively induced the luciferase activity in a theophylline-dependent manner, and it was also observed that the expression of the transgene was more effectively induced in the ribozyme to which a 300 nt anti-sense sequence was inserted than in the ribozyme to which a 100 nt anti-sense sequence was inserted.

5) Intracellular Trans-Splicing Reaction by Allosteric Ribozyme

The ribozyme constructs that can induce and enhance the transgene activity in a theophylline-dependent manner in the cells were searched in the above-mentioned experiment. In order to verify whether the induction of the theophylline-dependent transgene is induced by the allosteric effect of the intracellular trans-splicing reaction, 293 cells were transiently transfected with the expression vectors for the ribozymes to which a theophylline aptamer was attached, and the presence of the intracellular trans-splicing reaction product in the cells were observed.

An expression level of GAPDH RNA was observed, and used as the internal control. The RT-PCR product was analyzed on agarose gel. And the results are shown in FIG. 13.

Referring to FIG. 13, it was revealed that the hTERT-specific trans-splicing reaction product was produced in the positive control, WT ribozyme (AS-300 WT), as it was expected (Lane 3). For the ribozyme to which a theophylline aptamer was attached, it was observed that the trans-splicing product was produced from the AS-300 Mu-P9 6t8t ribozyme vectors regardless of the presence of theophylline, caffeine and PBS, which accords with the induction results of the luciferase activity (Lanes 7-9), but the 311 by trans-splicing product was produced only in the theophylline-treated cells, which accords with the induction results of the luciferase activity (Lane 4). These results were different from the in vitro trans-splicing results of the AS-300 W-P9 6t8t ribozyme, but it was considered that this difference is derived from the difference in the in vitro and intracellular environments in the cells. In order to verify that the trans-splicing product is produced by the intracellular trans-splicing reaction rather than the in vitro trans-splicing reaction induced in the RNA extraction procedure, the 293 cells and SK-Lu-1 cells (hTERT negative) transfected with the AS-300 W-P9 6t8t ribozyme vector were mixed, and RNA was extracted from the cells, and subjected to a RT-PCR reaction. As a result, since no trans-splicing product was observed (Lane 10), as shown in FIG. 13, the trans-splicing product measured at the presence of theophylline in the 293 cells transfected with the AS-300 W-P9 6t8t ribozyme was produced by the trans-splicing reaction that is dependent on the theophylline and specific to the target RNA in the cells.

As described above, the AS-300 W-P9 6t8t and AS-300 ΔP9 8 tribozymes have been developed as the candidates for allosteric ribozymes that can specifically control the expression of transgenes in a theophylline-dependent manner in the cells expressing the hTERT RNA, that is, can artificially control the RNA replacement reaction in a theophylline-dependent manner in the cells. In addition, the IGS W-P9 6t8t ribozyme have been developed as the allosteric ribozyme that can in vitro produce the trans-splicing product the most effectively.

### Example 5

### Observation of Functions to Control hTERT-Expressing Cancer Cell-Specific Apoptosis by Adenoviral Vector

Induction of Theophylline-Dependent Apoptosis in HT-29 Cells (hTERT+)

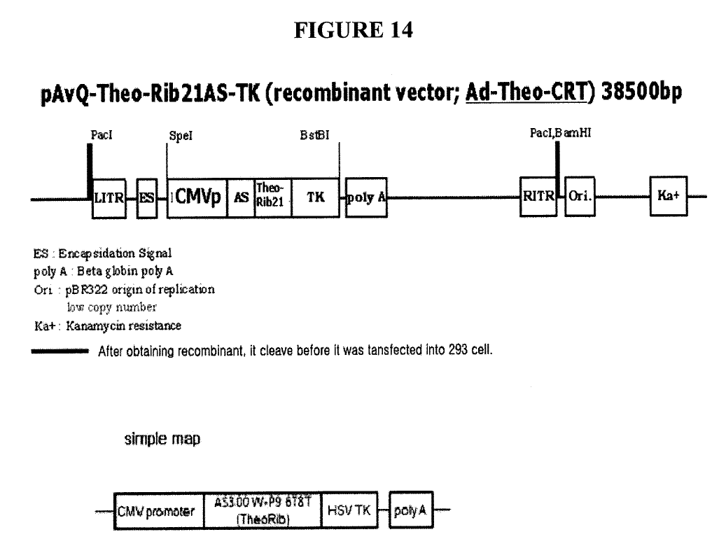

A vector (pAvQ-Theo-Rib21AS-TK, SEQ ID NO: 8) that can express ribozyme under the control of a CMV promoter in mammalian cells by inserting an apoptosis gene, HSV thymidine kinase, to a 3′ exon of the prepared allosteric ribozyme (AS300 W-P9 6T8T-TK), and a recombinant adenoviral vector was prepared (FIG. 14).

The pAvQ-Theo-Rib21AS-TK was deposited with Accession No. KCCM10935P in Korean Culture Center of Microorganisms (KCCM) on Mar. 21, 2008.

In order to observe whether the adenoviral vector (Ad-TheoRib-TK) contains an HSVtk gene as a 3′ exon and expresses theophylline-dependent allosteric ribozyme inducing transgene expression in target-specific and theophylline-dependent manners, the colon cancer cells, HT-29 cells, were treated with the adenoviral vector, treated with GCV and a regulator compound, and then subjected to an MTT assay to observe the cell viability of the HT-29 cells. In this case, Ad-TK (an adenoviral vector expressing an HSVtk gene under the control of a CMV promoter) was used as the positive control, and Ad-Rib-TK (an adenoviral vector that is specific to hTERT and tagged with HSVtk) was used as the positive control in the hTERT+ cells. Ad-LacZ (an adenoviral vector expressing a LacZ gene under the control of a CMV promoter) was used as the negative control. When the HT-29 cells were treated with Ad-TheoRib-TK, the cell viability of the HT-29 cells after the treatment of theophylline or caffeine was compared to that of the HT-29 cells treated with Ad-TheoRib-TK. The results are shown in FIG. 15.

Referring to FIG. 15, it was observed that the cell viability was decreased with the increases in the GCV concentration and the adenovirus concentration when the HT-29 cells were treated with the Ad-TK and Ad-Rib-TK, but the cell viability was not affected by the chemical concentration. Meanwhile, it was observed that the Ad-LacZ did not affect the cell viability at all. It was noted that, in the case of the HT-29 cells infected with the allosteric ribozyme Ad-TheoRib-TK, the cell viability was not affected by the increases in the concentrations of the GCV, viruses and chemicals when HT-29 cells were treated with caffeine, but the cell viability was decreased in proportion to the concentrations of viruses and GCV as in the positive control when the HT-29 cells were treated with theophylline. Also, it was observed that, when the concentration of theophylline was increased, the cell viability was also decreased with the increase in the concentration of theophylline. This indicates that the Ad-TheoRib-TK induced the apoptosis of the cancer cells since ribozyme activity was allosterically controlled by theophylline and the transgene expression was induced only when treated with theophylline. The optimum condition where the gene expression is allosterically induced is that the HT-29 cells were treated with 100 moi adenovirus, 100 μM theophylline and 10 μM GCV.

2) Induction of Theophylline-Dependent Apoptosis in HepG2 Cells (hTERT+)

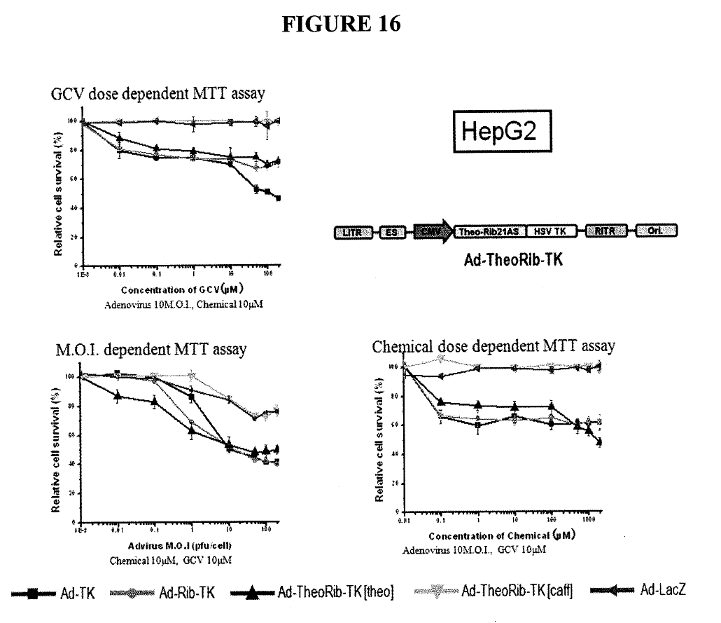

In order to observe whether the Ad-TheoRib-TK induces the transgene expression in target-specific and theophylline-dependent manners, the liver cancer cells, HepG2 cells, were treated with the adenoviral vector, treated with GCV and a regulator compound, and then subjected to an MTT assay to observe the cell viability in the HepG2 cells. In this case, Ad-TK was used as the positive control, and Ad-Rib-TK was used as the positive control in the hTERT+ cells. Ad-LacZ was used as the negative control. When the HepG2 cells were treated with Ad-TheoRib-TK, the cell viability of the HepG2 cells after the treatment of theophylline or caffeine was compared to that of the HepG2 cells treated with Ad-TheoRib-TK. The results are shown in FIG. 16.

Referring to FIG. 16, it was observed that the cell viability was decreased with the increases in the GCV and adenovirus concentrations when the HepG2 cells were treated with the Ad-TK and Ad-Rib-TK as observed in the HT-29 cells, but the cell viability was not affected by the chemical concentration. Meanwhile, it was observed that the Ad-LacZ did not affect the cell viability at all. It was noted that, in the case of the HepG2 cells infected with the allosteric ribozyme Ad-TheoRib-TK, the cell viability was not affected by the increases in the concentrations of the GCV, viruses and chemicals when the HepG2 cells were treated with caffeine, but the cell viability was decreased in proportion to the concentrations of viruses and GCV as in the positive control when the HepG2 cells were treated with theophylline. Also, it was observed that, when the concentration of theophylline was increased, the cell viability was also decreased with the increase in the concentration of theophylline. This indicates that the Ad-TheoRib-TK induced the apoptosis of the cancer cells in the HepG2 cells in addition to the hTERT+HT-29 cells since ribozyme activity was allosterically controlled by theophylline and the transgene expression was induced only when treated with theophylline. The optimum condition where the gene expression is allosterically induced is that the HepG2 cells were treated with 10 moi adenovirus, 10 μM theophylline and 10 μM GCV.

3) Induction Of Theophylline-Dependent Apoptosis In Capan-1 Cells (hTERT+)

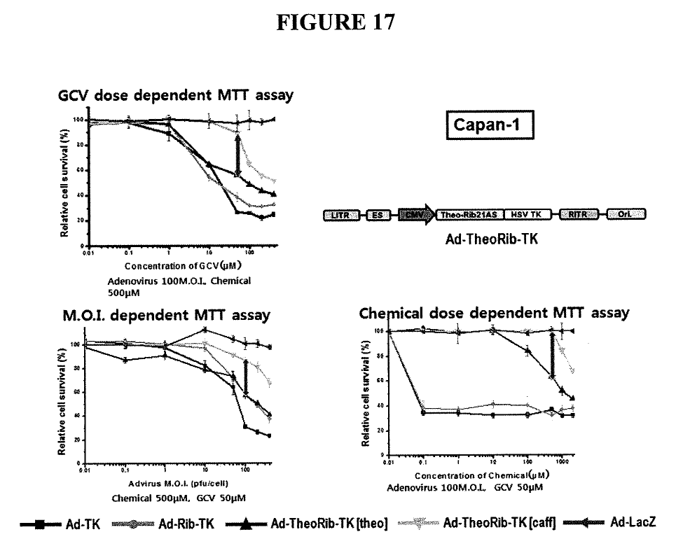

In order to observe whether the Ad-TheoRib-TK induces the transgene expression in target-specific and theophylline-dependent manners, the colon cancer cells(Capan-1 cells) were treated with the adenoviral vector, treated with GCV and a regulator compound, and then subjected to an MTT assay to observe the cell viability in the Capan-1 cells. The results are shown in FIG. 17.

Referring to FIG. 17, it was observed that the cell viability was decreased with the increases in the GCV concentration and the adenovirus concentration when the Capan-1 cells were treated with the Ad-TK and Ad-Rib-TK as observed in the HT-29 and HepG2 cells, but the cell viability was not affected by the chemical concentration. Meanwhile, it was observed that the Ad-LacZ did not affect the cell viability at all. It was noted that, in the case of the Capan-1 cells infected with the allosteric ribozyme Ad-TheoRib-TK, the cell viability was not affected by the increases in the concentrations of the GCV, virus and chemical when the Capan-1 cells were treated with caffeine, but the cell viability was decreased in proportion to the concentrations of virus and GCV as in the positive control when the Capan-1 cells were treated with theophylline. Also, it was observed that, when the concentration of theophylline was increased, the cell viability was also decreased with the increase in the concentration of theophylline. This indicates that the Ad-TheoRib-TK induced the apoptosis of the cancer cells in the Capan-1 cells in addition to the hTERT+HT-29 and HepG2 cells since ribozyme activity was allosterically controlled by theophylline and the transgene expression was induced only when treated with theophylline. The optimum condition where the gene expression is allosterically induced is that the Capan-1 cells were treated with 100 moi adenovirus, 500 μM theophylline and 50 μM GCV.

4) Observation of Induction of Theophylline-Dependent Apoptosis in IMR90 Cells (hTERT−)

In order to observe whether the theophylline-dependent control of the Ad-TheoRib-TK activity in the hTERT+ cells is specific to the target RNA, the hTERT−IMR90 cells were infected with the adenoviral vector, and the cell viability in the hTERT−IMR90 cells were then observed. The results are shown in FIG. 18.

Referring to FIG. 18, it was observed that the cell viability was decreased regardless of the concentrations of the adenovirus and GCV when the hTERT−IMR90 cells were treated with the Ad-TK. This indicates that the results have nothing to do with the chemical concentration. However, the Ad-Rib-TK expressing the ribozyme and the allosteric Ad-TheoRib-TK may not affect the cell viability regardless of the concentrations of virus, GCV and chemical even when the concentrations of the Ad-Rib-TK and the allosteric Ad-TheoRib-TK were increased. This indicates that the Ad-TheoRib-TK may artificially control the activity of the ribozyme at the presence of the exogenous compound, and also induce the transgene in a highly target-specific manner.

### Example 6

### Control of Theophylline-Dependent Intracellular Trans-Splicing Reaction by Allosteric Ribozyme-Expressing Adenoviral Vector

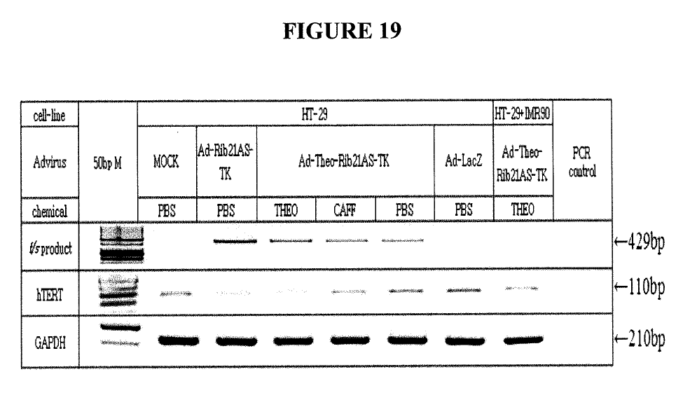

In order to verify whether the induction of the theophylline-dependent transgene is induced by the allosteric effects of the intracellular trans-splicing reaction, HT-29 cells were infected with the adenoviral vector (100 moi) expressing the ribozyme to which a theophylline aptamer is attached, and then treated with 0.1 mM theophylline or an equivalent concentration of caffeine which is the optimum condition established in this experiment to observe whether the intracellular trans-splicing reaction product is produced in the cells. An expression level of GAPDH RNA was observed, and then used as the internal control. The RT-PCR product was analyzed on agarose gel, and the results are shown in FIG. 19.

Referring to FIG. 19, it was observed that any trans-splicing product was not produced regardless of the treatment with a small molecule compound in the case of the negative control Ad-LacZ, as it was expected. Meanwhile, when the Ad-TheoRib-TK (Ad-Theo-Rib2AS-TK) was introduced into the cancer cells, HT-29 cells, that express the hTERT, and treated with caffeine, the trans-splicing product was hardly produced, but the expected 429 nt trans-splicing product was produced when the HT-29 cells were treated with 0.1 mM theophylline, which accords with the results observed in the MTT assay. When the trans-splicing product was cloned and sequenced, it was observed that a +21st site of the hTERT was spliced in the trans-splicing product. Meanwhile, the trans-splicing product was not produced in the IMR90 cell that does not express the hTERT under the same condition as described above, which indicate that the ribozyme according to the present invention shows its trans-splicing function only in the presence of the target RNA. In order to verify that the theophylline dependent trans-splicing product is not an in vitro trans-splicing reaction induced in the RNA extraction procedure but an intracellular trans-splicing reaction, the mock-transfected HT-29 cells and the IMR90 cells (hTERT negative) transfected with Ad-TheoRib-TK and treated with theophylline were mixed, and RNA was then extracted from the cell mixture, and subjected to a RT-PCR reaction (mix). As a result, the expected trans-splicing product was not observed in the cell mixture, which indicates that the trans-splicing product and apoptosis as measured only in the Ad-TheoRib-TK-introduced HT-29 cells in the presence of theophylline was induced by the theophylline-dependent and target RNA-specific trans-splicing reaction.

In order to compare a relative concentration of the trans-splicing reaction product in the HT-29 cells, the allosteric trans-splicing ribozyme was reversely transcribed, and then subjected to a real-time PCR. A concentration of the T/S PCR product was corrected with the concentration of the GAPDH PCR product, and plotted in graph (FIG. 20).

Referring to FIG. 20, it is observed that any trans-splicing product was not produced regardless of the treatment with a small molecule compound in the case of the negative control Ad-LacZ. Meanwhile, when the Ad-TheoRib-TK was introduced into the cells, and then treated with PBS, the trans-splicing product was hardly produced, and, when the Ad-TheoRib-TK was treated with caffeine, a concentration of the reaction product was more slightly increased than when the Ad-TheoRib-TK was treated with PBS, but more significantly decreased by 78% than when Ad-TheoRib-TK was treated with theophylline. Meanwhile, when the Ad-TheoRib-TK was introduced into the cells, and then treated with theophylline, the trans-splicing reaction was effectively induced to a concentration as much as the trans-splicing product produced by the Ad-Rib-TK. This result indicates that the induction of the theophylline-dependent and target-specific apoptosis induced by the allosteric ribozyme owes itself to the activation of the target-specific trans-splicing reaction by theophylline.

### INDUSTRIAL APPLICABILITY

As described above, the present invention is based on the combination of a very specific gene therapy and a trans-splicing ribozyme, that can target disease-specific RNA and induce gene expression by establishing, as a model system, trans-splicing ribozymes whose activities can be controlled by theophylline, and the reversible genetic technology where the gene expression can be controlled by the trans-splicing ribozyme and exogenous factors. The allosteric trans-splicing group I ribozyme according to one exemplary embodiment of the present invention may be used as a common gene therapeutic agent that may be used to treat a variety of incurable diseases, and also used as a tool to develop a diagnostic agent, or as a mechanism to search for the activity mechanism of the ribozyme.

What is claimed is:

1. A method for selecting an allosteric trans-splicing group I ribozyme whose activity is controlled by theophylline, the method comprising:
preparing an aptazyme where a theophylline aptamer and a communication module bind to either or both of P6 and P8 domains of a trans-splicing ribozyme, an aptazyme where a theophylline aptamer and a communication module bind to either or both of P6 and P8 domains of a trans-splicing ribozyme whose P9 domain is partially removed, or an aptazyme where a theophylline aptamer and a communication module bind to either or both of P6 and P8 domains of a trans-splicing ribozyme whose P9 domain is partially modified;
confirming whether a theophylline-dependent trans-splicing reaction occurs by using theophylline and caffeine to compare the allosteric controls of the in vitro prepared aptazyme; and
confirming whether a theophylline-dependent transgene is expressed at the presence of 0.1 to 1 mM theophylline by luciferase activity in mammalian cells.

2. The method for selecting an allosteric trans-splicing group I ribozyme according to claim 1, further comprising: preparing an aptazyme including an anti-sense 100 to 300 nt segment against hTERT RNA in the step of preparing an aptazyme.

3. The method for selecting an allosteric trans-splicing group I ribozyme according to claim 1, wherein the modified P9 domain of the trans-splicing ribozyme has a DNA sequence of ‘CGAAAGGGAG’.

4. An allosteric trans-splicing group I ribozyme whose RNA replacement activity is controlled by theophylline, characterized in that the allosteric trans-splicing group I ribozyme specifically targets RNA of human Telomerase reverse transcriptase (hTERT), and has a firefly-derived luciferase receptor gene at 3′ exon.

5. The allosteric trans-splicing group I ribozyme according to claim 4, wherein the ribozyme has a RNA sequence selected from the group consisting of AS300 ΔP9 8T set forth in SEQ ID NO: 1, AS100 Mu-P9 6T8T set forth in SEQ ID NO: 2 and AS300 W-P9 6T8T set forth in SEQ ID NO: 3.

6. An expression vector encoding the allosteric trans-splicing group I ribozyme defined in claim 4.

7. The expression vector according to claim 6, wherein the expression vector comprises a vector selected from the group consisting of pSEAP AS300 Delta P9 8T-Luci set forth in SEQ ID NO: 4, pSEAP AS100 Mu-P9 6T8T-Luci set forth in SEQ ID NO: 5 and pSEAP AS300 W-P9 6T8T-Luci set forth in SEQ ID NO: 6.

8. An allosteric trans-splicing group I ribozyme whose RNA replacement activity is controlled by theophylline, characterized in that the allosteric trans-splicing group I ribozyme specifically targets RNA of human Telomerase reverse transcriptase (hTERT), and has a herpes simplex virus thymidine kinase (HSV-TK) apoptosis gene at 3′ exon.

9. The allosteric trans-splicing group I ribozyme according to claim 8, wherein the ribozyme has an RNA sequence of AS300 W-P9 6T8T-TK set forth in SEQ ID NO: 7.

10. An expression vector expressing the allosteric trans-splicing group I ribozyme defined in claim 8 in mammalian cells.

11. The expression vector according to claim 10, wherein the expression vector comprises pAvQ-Theo-Rib21AS-TK (KCCM 10935P) set forth in SEQ ID NO: 8.

12. A gene expression inducer, comprising theophylline and the allosteric trans-splicing group I ribozyme defined in claim 4 or 8.

13. A gene expression inducer, comprising theophylline and the expression vector defined in claim 6 or 10.

14. A cancer diagnostic agent, comprising theophylline and the allosteric trans-splicing group I ribozyme defined in claim 4 or 8.

15. A cancer diagnostic agent, comprising theophylline and the expression vector defined in claim 6 or 10.

16. A gene therapeutic agent, comprising theophylline and the allosteric trans-splicing group I ribozyme defined in claim 4 or 8.

17. A gene therapeutic agent, comprising theophylline and the expression vector defined in claim 6 or 10.


SEQUENCE LISTING (39 sequences)

SEQ ID NO: 1 - RNA, 2347 nt, Artificial Sequence
  allosteric trans splicing group I ribozyme AS300 Delta P9 8T
aaggccagcacguucuucgcgccgcgcucgcacagccucugcagcacucgggccaccagcuccuucaggcaggacaccuggcggaaggagggggcggcggggggcggccgugcgucccagggcacgcacaccaggcacugggccaccagcgcgcggaaagccgccggguccccgcgcugcaccagccgccagcccuggggccccaggcgccgcacgaacguggccagcggcagcaccucgcgguaguggcugcgcagcagggagcgcacggcuaggcagcggggagcgcgcggcaucgcggggguggccggggccagggcuucccaagcuucguuuugcggcaggaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagucaacagcaugcacuguugauauggaugcaguucacagacuaaaugucggucggggaugauaccagccgaaaggcccuuggcagcaaucauaagauauagucggaccucucccgaaagggaguuggaaguacucgcgaaaacgcccaccauggaagacgccaaaaacauaaagaaaggcccggcgccauucuauccucuagaggauggaaccgcuggagagcaacugcauaaggcuaugaagagauacgcccugguuccuggaacaauugcuuuuacagaugcacauaucgaggugaacaucacguacgcggaauacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugaacauuucgcagccuaccguaguguuuguuuccaaaaagggguugcaaaaaauuuugaacgugcaaaaaaaauuaccaauaauccagaaaauuauuaucauggauucuaaaacggauuaccagggauuucagucgauguacacguucgucacaucucaucuaccucccgguuuuaaugaauacgauuuuguaccagaguccuuugaucgugacaaaacaauugcacugauaaugaauuccucuggaucuacuggguuaccuaaggguguggcccuuccgcauagaacugccugcgucagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuuuuacgaucccuucaggauuacaaaauucaaagugcguugcuaguaccaacccuauuuucauucuucgccaaaagcacucugauugacaaauacgauuuaucuaauuuacacgaaauugcuucugggggcgcaccucuuucgaaagaagucggggaagcgguugcaaaacgcuuccaucuuccagggauacgacaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucagagaggcgaauuaugugucagaggaccuaugauuauguccgguuauguaaacaauccggaagcgaccaacgccuugauugacaaggauggauggcuacauucuggagacauagcuuacugggacgaagacgaacacuucuucauaguugaccgcuugaagucuuuaauuaaauacaaaggauaucagguggcccccgcugaauuggaaucgauauuguuacaacaccccaacaucuucgacgcgggcguggcaggucuucccgacgaugacgccggugaacuucccgccgccguuguuguuuuggagcacggaaagacgaugacggaaaaagagaucguggauuacguggccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaaguccaaauuguaa

SEQ ID NO: 2 - RNA, 2360 nt, Artificial Sequence
  allosteric trans-splicing group I ribozyme AS100 Mu-P9 6T8T
aaggccagcacguucuucgcgccgcgcucgcacagccucugcagcacucgggccaccagcuccuucaggcaggacaccuggcggaaggagggggcggcggggggcggccgugcgucccagggcacgcacaccaggcacugggccaccagcgcgcggaaagccgccggguccccgcgcugcaccagccgccagcccuggggccccaggcgccgcacgaacguggccagcggcagcaccucgcgguaguggcugcgcagcagggagcgcacggcuaggcagcggggagcgcgcggcaucgcggggguggccggggccagggcuucccaagcuucguuuugcggcaggaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagggaugauaccagccgaaaggcccuuggcagcaauuauggaugcaguucacagacuaaaugucggucggggaugauaccagccgaaaggcccuuggcagcaaucauaagauauagucggaccucucccgaaagggaguuggaguacucgcgaaaacgcccaccauggaagacgccaaaaacauaaagaaaggcccggcgccauucuauccucuagaggauggaaccgcuggagagcaacugcauaaggcuaugaagagauacgcccugguuccuggaacaauugcuuuuacagaugcacauaucgaggugaacaucacguacgcggaauacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugaacauuucgcagccuaccguaguguuuguuuccaaaaagggguugcaaaaaauuuugaacgugcaaaaaaaauuaccaauaauccagaaaauuauuaucauggauucuaaaacggauuaccagggauuucagucgauguacacguucgucacaucucaucuaccucccgguuuuaaugaauacgauuuuguaccagaguccuuugaucgugacaaaacaauugcacugauaaugaauuccucuggaucuacuggguuaccuaaggguguggcccuuccgcauagaacugccugcgucagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuuuuacgaucccuucaggauuacaaaauucaaagugcguugcuaguaccaacccuauuuucauucuucgccaaaagcacucugauugacaaauacgauuuaucuaauuuacacgaaauugcuucugggggcgcaccucuuucgaaagaagucggggaagcgguugcaaaacgcuuccaucuuccagggauacgacaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucagagaggcgaauuaugugucagaggaccuaugauuauguccgguuauguaaacaauccggaagcgaccaacgccuugauugacaaggauggauggcuacauucuggagacauagcuuacugggacgaagacgaacacuucuucauaguugaccgcuugaagucuuuaauuaaauacaaaggauaucagguggcccccgcugaauuggaaucgauauuguuacaacaccccaacaucuucgacgcgggcguggcaggucuucccgacgaugacgccggugaacuucccgccgccguuguuguuuuggagcacggaaagacgaugacggaaaaagagaucguggauuacguggccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaaguccaaauuguaa

SEQ ID NO: 3 - RNA, 2437 nt, Artificial Sequence
  allosteric trans-splicing group I ribozyme AS300 W-P9 6T8T
aagccgaaggccagcacguucuucgcgccgcgcucgcacagccucugcagcacucgggccaccagcuccuucaggcaggacaccuggcggaaggagggggcggcggggggcggccgugcgucccagggcacgcacaccaggcacugggccaccagcgcgcggaaagccgccggguccccgcgcugcaccagccgccagcccuggggccccaggcgccgcacgaacguggccagcggcagcaccucgcgguaguggcugcgcagcagggagcgcacggcuaggcagcggggagcgcgcggcaucgcggggguggccggggccagggcuucccaagcuucguuuugcggcaggaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagggaugauaccagccgaaaggcccuuggcagcaauuauggaugcaguucacagacuaaaugucggucggggaugauaccagccgaaaggcccuuggcagcaaucauaagauauagucggaccucuccuuaaugggagcuagcggaugaagugaugcaacacuggagccgcugggaacuaauuuguaugcgaaaguauauugauuaguuuuggaguacucgcgaaaacgcccaccauggaagacgccaaaaacauaaagaaaggcccggcgccauucuauccucuagaggauggaaccgcuggagagcaacugcauaaggcuaugaagagauacgcccugguuccuggaacaauugcuuuuacagaugcacauaucgaggugaacaucacguacgcggaauacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugaacauuucgcagccuaccguaguguuuguuuccaaaaagggguugcaaaaaauuuugaacgugcaaaaaaaauuaccaauaauccagaaaauuauuaucauggauucuaaaacggauuaccagggauuucagucgauguacacguucgucacaucucaucuaccucccgguuuuaaugaauacgauuuuguaccagaguccuuugaucgugacaaaacaauugcacugauaaugaauuccucuggaucuacuggguuaccuaaggguguggcccuuccgcauagaacugccugcgucagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuuuuacgaucccuucaggauuacaaaauucaaagugcguugcuaguaccaacccuauuuucauucuucgccaaaagcacucugauugacaaauacgauuuaucuaauuuacacgaaauugcuucugggggcgcaccucuuucgaaagaagucggggaagcgguugcaaaacgcuuccaucuuccagggauacgacaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucagagaggcgaauuaugugucagaggaccuaugauuauguccgguuauguaaacaauccggaagcgaccaacgccuugauugacaaggauggauggcuacauucuggagacauagcuuacugggacgaagacgaacacuucuucauaguugaccgcuugaagucuuuaauuaaauacaaaggauaucagguggcccccgcugaauuggaaucgauauuguuacaacaccccaacaucuucgacgcgggcguggcaggucuucccgacgaugacgccggugaacuucccgccgccguuguuguuuuggagcacggaaagacgaugacggaaaaagagaucguggauuacguggccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaaguccaaauuguaa

SEQ ID NO: 4 - DNA, 5674 bp, Artificial Sequence
  AS300 Delta P9 8T expression vector(pSEAP AS300 Delta P9 8T-Luci)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagcatgcactgttgatatggatgcagttcacagactaaatgtcggtcggggatgataccagccgaaaggcccttggcagcaatcataagatatagtcggacctctcccgaaagggagttggaagtactcgcgaaaacgcccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtggccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 5 - DNA, 5687 bp, Artificial Sequence
  AS100 Mu-P9 6T8T expression vector (pSEAP AS100 Mu-P9 6T8T-Luci)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagggatgataccagccgaaaggcccttggcagcaattatggatgcagttcacagactaaatgtcggtcggggatgataccagccgaaaggcccttggcagcaatcataagatatagtcggacctctcccgaaagggagttggagtactcgcgaaaacgcccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtggccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 6 - DNA, 5764 bp, Artificial Sequence
  AS300 W-P9 6T8T expression vector (pSEAP AS300 W-P9 6T8T-Luci)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttaagccgaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagggatgataccagccgaaaggcccttggcagcaattatggatgcagttcacagactaaatgtcggtcggggatgataccagccgaaaggcccttggcagcaatcataagatatagtcggacctctccttaatgggagctagcggatgaagtgatgcaacactggagccgctgggaactaatttgtatgcgaaagtatattgattagttttggagtactcgcgaaaacgcccaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtggccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - RNA, 1910 nt, Artificial Sequence
  allosteric trans-splicing group I ribozyme AS 300 W-P9 6T8T-TK
aaggccagcacguucuucgcgccgcgcucgcacagccucugcagcacucgggccaccagcuccuucaggcaggacaccuggcggaaggagggggcggcggggggcggccgugcgucccagggcacgcacaccaggcacugggccaccagcgcgcggaaagccgccggguccccgcgcugcaccagccgccagcccuggggccccaggcgccgcacgaacguggccagcggcagcaccucgcgguaguggcugcgcagcagggagcgcacggcuaggcagcggggagcgcgcggcaucgcggggguggccggggccagggcuucccaagcuucguuuugcggcaggaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagggaugauaccagccgaaaggcccuuggcagcaauuauggaugcaguucacagacuaaaugucggucggggaugauaccagccgaaaggcccuuggcagcaaucauaagauauagucggaccucuccuuaaugggagcuagcggaugaagugaugcaacacuggagccgcugggaacuaauuuguaugcgaaaguauauugauuaguuuuggaguacucgaaaacgcccaccauggcuucguaccccugccaucaacacgcgucugcguucgaccaggcugcgcguucucgcggccauagcaaccgacguacggcguugcgcccucgccggcagcaagaagccacggaaguccgccuggagcagaaaaugcccacgcuacugcggguuuauauagacgguccucacgggauggggaaaaccaccaccacgcaacugcugguggcccuggguucgcgcgacgauaucgucuacguacccgagccgaugacuuacuggcaggugcugggggcuuccgagacaaucgcgaacaucuacaccacacaacaccgccucgaccagggugagauaucggccggggacgcggcggugguaaugacaagcgcccagauaacaaugggcaugccuuaugccgugaccgacgccguucuggcuccucaugucgggggggaggcugggaguucacaugccccgcccccggcccucacccucaucuucgaccgccaucccaucgccgcccuccugugcuacccggccgcgcgauaccuuaugggcagcaugaccccccaggccgugcuggcguucguggcccucaucccgccgaccuugcccggcacaaacaucguguugggggcccuuccggaggacagacacaucgaccgccuggccaaacgccagcgccccggcgagcggcuugaccuggcuaugcuggccgcgauucgccgcguuuacgggcugcuugccaauacggugcgguaucugcagggcggcgggucguggugggaggauuggggacagcuuucggggacggccgugccgccccagggugccgagccccagagcaacgcgggcccacgaccccauaucggggacacguuauuuacccuguuucgggcccccgaguugcuggcccccaacggcgaccuguauaacguguuugccugggccuuggacgucuuggccaaacgccuccgucccaugcacgucuuuauccuggauuacgaccaaucgcccgccggcugccgggacgcccugcugcaacuuaccuccgggaugguccagacccacgucaccaccccaggcuccauaccgacgaucugcgaccuggcgcgcacguuugcccgggagaugggggaggcuaacugauua

SEQ ID NO: 8 - DNA, 9996 bp, Artificial Sequence
  AS300 W-P9 6T8T-TK expression vector (pAvQ-Theo-Rib21AS-TK)
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagccagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagccggcggatggtcgaggtgaggtgtggcaggcttgagatccagctgttggggtgagtactccctctcaaaagcgggcatgacttctgcgctaagattgtcagtttccaaaaacgaggaggatttgatattcacctggcccgatctggccatacacttgagtgacaatgacatccactttgcctttctctccacaggtgtccactcccaggtccaagtttggaagatccaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagggatgataccagccgaaaggcccttggcagcaattatggatgcagttcacagactaaatgtcggtcggggatgataccagccgaaaggcccttggcagcaatcataagatatagtcggacctctccttaatgggagctagcggatgaagtgatgcaacactggagccgctgggaactaatttgtatgcgaaagtatattgattagttttggagtactcgaaaacgcccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgattcgaaagatcccaacgaaaagagagaccacatggtccttcttgagtttgtaacagctgctgggattacacatggcatggatgaactgtacaactgaggatcccccgacctcgacctctggctaataaaggaaatttattttcattgcaatagtgtgttggaattttttgtgtctctcactcggaaggacatatgggagggcaaatcatttggtcgagatccctcggagatcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaat

SEQ ID NO: 9 - DNA, 47 bp, Artificial Sequence
  primer
ggggaattctaatacgactcactatagggcaggcagcgctgcgtcct

SEQ ID NO: 10 - DNA, 32 bp, Artificial Sequence
  primer
cgggatccctggcggaaggagggggcggcggg

SEQ ID NO: 11 - DNA, 47 bp, Artificial Sequence
  primer
ggggaattctaatacgactcactataggcaggaaaagttatcaggca

SEQ ID NO: 12 - DNA, 22 bp, Artificial Sequence
  primer
cgagtactccaaaactaatcaa

SEQ ID NO: 13 - DNA, 18 bp, Artificial Sequence
  primer
cgatgatcacgaagacgc

SEQ ID NO: 14 - DNA, 37 bp, Artificial Sequence
  primer
aaggaaaaaagcggccgcttattacaatttggacttt

SEQ ID NO: 15 - DNA, 32 bp, Artificial Sequence; identical to SEQ ID NO: 10
  primer

SEQ ID NO: 16 - DNA, 47 bp, Artificial Sequence; identical to SEQ ID NO: 11
  primer

SEQ ID NO: 17 - DNA, 30 bp, Artificial Sequence
  primer
cccaagcttgcgcaactgcaactccgataa

SEQ ID NO: 18 - DNA, 30 bp, Artificial Sequence; identical to SEQ ID NO: 17
  primer

SEQ ID NO: 19 - DNA, 25 bp, Artificial Sequence
  primer
ggaattcgcagcgctgcgtcctgct

SEQ ID NO: 20 - DNA, 27 bp, Artificial Sequence
  primer
cccaagctttcactgcatacgacgatt

SEQ ID NO: 21 - DNA, 37 bp, Artificial Sequence
  primer
gcccaacaccggcataaagttacataattacacactt

SEQ ID NO: 22 - DNA, 23 bp, Artificial Sequence
  primer
cccgaattctgcgtcctgctcga

SEQ ID NO: 23 - DNA, 26 bp, Artificial Sequence
  primer
cccaagctttcactgcatacacgatt

SEQ ID NO: 24 - DNA, 18 bp, Artificial Sequence
  primer
atgactgaatataaactt

SEQ ID NO: 25 - DNA, 26 bp, Artificial Sequence
  primer
cccaagctttacataattacacactt

SEQ ID NO: 26 - DNA, 45 bp, Artificial Sequence
  primer
aattcaagcttcgttttgcggcagcaggaaaagttatcaggcatg

SEQ ID NO: 27 - DNA, 34 bp, Artificial Sequence
  primer
cctgataacttttcctgccgcaaaacgaagcttg

SEQ ID NO: 28 - DNA, 24 bp, Artificial Sequence
  primer
gggaagcttgggaagccctggccc

SEQ ID NO: 29 - DNA, 26 bp, Artificial Sequence
  primer
gggaagcttaaggccagcacgttctt

SEQ ID NO: 30 - DNA, 30 bp, Artificial Sequence; identical to SEQ ID NO: 17
  primer

SEQ ID NO: 31 - DNA, 28 bp, Artificial Sequence
  primer
cccaagcttgcccaacaccggcataaag

SEQ ID NO: 32 - DNA, 16 bp, Artificial Sequence
  primer
agcgctgcgtcctgct

SEQ ID NO: 33 - DNA, 20 bp, Artificial Sequence
  primer
tgacatcaagaaggtggtga

SEQ ID NO: 34 - DNA, 20 bp, Artificial Sequence
  primer
tccaccaccctgttgctgta

SEQ ID NO: 35 - DNA, 24 bp, Artificial Sequence
  primer
cccatgcacgtctttatcctggat

SEQ ID NO: 36 - DNA, 25 bp, Artificial Sequence
  primer
ggaattcgcagcgctgcgtcctgct

SEQ ID NO: 37 - DNA, 20 bp, Artificial Sequence
  primer
tgacatcaagaaggtggtga

SEQ ID NO: 38 - DNA, 20 bp, Artificial Sequence
  primer
tccaccaccctgttgctgta

SEQ ID NO: 39 - DNA, 47 bp, Artificial Sequence; identical to SEQ ID NO: 11
  primer